(19) United States
(12) Patent Application Publication
(10) Pub. No.: US 2019/0321374 A1
(43) Pub. Date: Oct. 24, 2019

(54) METHODS FOR IMPROVING QUALITY OF LIFE OR SEXUAL DOMAIN FUNCTION AND COMPOSITION USEFUL IN THE METHODS

(71) Applicant: Lipocine Inc., Salt Lake City, UT (US)

(72) Inventors: Mahesh V. Patel, Salt Lake City, UT (US); Nachiappan Chidambaram, Sandy, UT (US); Satish Nachaegari, Salt Lake City, UT (US)

(73) Assignee: Lipocine Inc., Salt Lake City, UT (US)

(21) Appl. No.: 16/195,763

(22) Filed: Nov. 19, 2018

Related U.S. Application Data
(63) Continuation of application No. 15/348,680, filed on Nov. 10, 2016, now abandoned.
(60) Provisional application No. 62/256,612, filed on Nov. 17, 2015.
(60) Provisional application No. 62/253,438, filed on Nov. 10, 2015.
(60) Provisional application No. 62/253,237, filed on Nov. 10, 2015.

(51) Int. Cl. A61K 31/568; A61K 47/10; A61K 47/44; A61K 9/107; A61K 9/00; A61K 9/48; A61K 47/14

(52) U.S. Cl. CPC A61K 31/568; A61K 47/10; A61K 47/44; A61K 47/14; A61K 9/0053; A61K 9/4858; A61K 9/107

(57) ABSTRACT

This application relates to methods and compositions useful for improving quality of life, sexual domain function or a combination thereof.

### PRIORITY DATA

This application claims the benefit of U.S. Provisional Application Ser. Nos. 62/256,612, filed Nov. 17, 2015; 62/253,438, filed Nov. 10, 2015; 62/253,237, filed Nov. 10, 2015 each of which is incorporated herein by reference in its entirety.

### FIELD OF THE INVENTION

Described herein are methods of treatment with pharmaceutical compositions and dosage forms. Accordingly, the present disclosure generally involves health sciences the fields of chemistry, pharmaceutical sciences and medicine.

### BACKGROUND OF THE INVENTION

A number of testosterone replacement therapies (TRT or TRTs) are currently marketed in the United States. None of the FDA approved TRTs are administered orally. Development Oral TRT is particularly challenging given the inherent difficulties of meeting FDA requirements for safety and efficacy (e.g., based on serum testosterone levels) coupled with a drugs such as testosterone esters. The approved TRTs are injectable or transdermal/transmucosal formulations. Thus, there is a need for oral formulations that meet FDA efficacy and safety guidelines for serum testosterone levels while improving quality of life or sexual function measures.

### SUMMARY OF THE INVENTION

The present disclosure is related to formulations and methods of orally administering formulations containing a lipophilic drug like testosterone undecanoate or other testosterone esters.

It was surprisingly discovered that oral testosterone undecanoate compositions can be administered to a male and improve one or more measures of quality of life such as physical role (e.g., role physical), vitality, social functioning, mental health and mental component summary in those males with the SF-36 instrument or a corollary thereof. Additionally, the oral testosterone undecanoate compositions when administered to a male and improve one or more measures of sexual domain function. Thus, provided herein are testosterone undecanoate formulations which are administered orally to males (e.g., hypogonadal, testosterone deficient or having a symptom thereof) and provide improvements to quality of life, sexual domain function or a combination thereof. For example, the methods and compositions can improve one or more measures of quality of life such as physical role (e.g., role physical), vitality, social functioning, mental health and mental component (as measured via the SF-36 questionnaire or any other appropriate or analogous quality of life measurement) in those males as well as improving sexual domain function. The orally administered compositions and associated methods were better than those for Androgel in some aspects, including statistically significant improvements over Androgel.

Given this information, the ordinary skilled artisan is now capable of providing oral testosterone undecanoate formulations that are bioequivalent for serum testosterone, testosterone undecanoate, dihydrotestosterone, dihydrotestosterone undecanoate or a combination thereof to the formulation used in this study for Cₘₐₓ, AUC₍₀₋ₜ₎, AUC_{(0-∞)}, C_{avg}, or a combination thereof, which are administered orally to males (e.g., hypogonadal, testosterone deficient or having a symptom thereof) and provide improvements to quality of life, sexual domain function or a combination thereof. Pharmaceutically equivalent formulations are also provided that are bioequivalent as described in this paragraph and elsewhere in this specification.

Described herein is a pharmaceutical composition that is bioequivalent for serum testosterone levels to a formulation having 50 mg to 350 mg testosterone undecanoate (e.g., in one aspect either 75 mg or 112.5 mg TU) at about 15% loading, about 63% Maisine 35-1, about 16% Cremophor RH 40 and about 6% PEG 8000. The bioequivalent composition typically has from 10%-50% testosterone undecanoate and 50%-90% pharmaceutically acceptable carrier. Moreover, the bioequivalent composition is bioequivalent for serum testosterone, testosterone undecanoate, dihydrotestosterone, dihydrotestosterone undecanoate or a combination thereof, for Cₘₐₓ, AUC₍₀₋ₜ₎, AUC_{(0-∞)}, C_{avg}, or a combination thereof at low, standard, and high fat conditions as well as standard versus low fat, standard versus high fat and low versus high fat. Any carrier can be used so long as the composition is bioequivalent to the formulation having 50 mg to 350 mg testosterone undecanoate (e.g., in one aspect either 75 mg, 112.5 mg, 150 mg, 225 mg or 350 mg TU) at about 15% loading, about 63% Maisine 35-1, about 16% Cremophor RH 40 and about 6% PEG 8000. In some aspects, the formulation is also pharmaceutically equivalent to that described herein. The pharmaceutical compositions when administered orally to a male provides an improvement to quality of life, sexual domain function or a combination thereof. The male can be desiring, or in need of, improvement in sexual domain function or quality of life. In specific implementations the male can be desiring, or in need of, improvement in sexual domain function or quality of life, can be hypogonadal, testosterone deficient or have a symptom thereof.

Also provided herein is a method for replacement therapy in a male for conditions associated with a deficiency or absence of endogenous testosterone provides an improvement to quality of life, sexual domain function or a combination thereof. The method can involve orally administering a pharmaceutical composition having TU and a pharmaceutically acceptable carrier, with a meal, to a male having a condition associated with a deficiency or absence of endogenous testosterone. The meal can be any meal regardless of fat content, the meal can also be a low fat meal, a standard fat meal or a high fat meal. The method provides bioequivalent Cₘₐₓ, AUC₍₀₋ₜ₎, AUC_{(0-∞)}, C_{avg}, or a combination thereof, when administered with low fat, standard fat and high fat meals. Alternatively, the method provides bioequivalent Cₘₐₓ, AUC₍₀₋ₜ₎, AUC_{(0-∞)}, C_{avg}, or a combination thereof, when administered with a standard fat meal as compared to low and high fat meals. In another alternative, the method provides bioequivalent Cₘₐₓ, AUC₍₀₋ₜ₎, AUC_{(0-∞)}, C_{avg}, or a combination thereof when administered with a low fat as compared to a high fat meal. When the composition is administered with a TU total daily dose range of about 275 mg (e.g., 300 mg) to about 625 mg (e.g., 600 mg), provides a serum testosterone C_{avg} at steady state in the range of 300 ng/dL to about 1100 ng/dL to a subject in need of treatment. The method can include a dose titration as described herein.

It is noted that the drug label can indicate (or method involve) that the oral testosterone replacement therapy (testosterone undecanoate containing oral dosage form) is taken (1) “WITH A MEAL” or (2) “WITH MEAL, BUT NOT ON EMPTY STOMACH” or (3) “WITH FAT CONTAINING FOOD” not specifying fat content. In an alternative, the drug label may indicate (or method may involve) the oral testosterone replacement therapy (testosterone undecanoate containing oral dosage form) is taken “WITH MEAL, BUT NOT LOW FAT”. In an alternative, the drug label may indicate (or method may involve) the oral testosterone replacement therapy (testosterone undecanoate containing oral dosage form) is taken “WITH MEAL, BUT NOT HIGH FAT”. In an alternative, the drug label may indicate (or method may involve) the oral testosterone replacement therapy (testosterone undecanoate containing oral dosage form) is taken “WITH STANDARD OR NORMAL MEAL”. Therefore, the oral testosterone undecanoate composition can be administered to a hypogonadal male (e.g., a male having testosterone deficiency or a symptom thereof; a male in need of testosterone replacement therapy) under the meal conditions described in this paragraph and improve one or more measures of quality-of-life such as physical role (e.g., role physical), vitality, social functioning, mental health and mental component in those males. Additionally, the oral testosterone undecanoate compositions when administered under a meal condition described in this paragraph to a male (e.g., having testosterone deficiency or a symptom thereof) and improve one or more measures of sexual domain function.

### BRIEF DESCRIPTION OF THE DRAWINGS

FIG. 1: Schematic illustration of clinical trial, see Example 1.

FIG. 2: Summary of results of sexual function questionnaire, see Example 1.

FIG. 3: Summary of results of quality of life questionnaire, see Example 1.

FIG. 4: Summary of results for free testosterone levels, see Example 4.

FIG. 5: Summary of results for SHBG levels, see Example 4.

FIG. 6: Summary of results for quality of life improvements in patients having one or more psychiatric disorders, see Example 4.

### DETAILED DESCRIPTION

Described herein are methods and compositions for improving quality of life, sexual domain function or a combination thereof. In specific implementations, the compositions and methods are useful for treatment of a hypogonadal male (or a symptom thereof) or male having testosterone deficiency (or a symptom thereof). In other specific implementations, the compositions and methods are useful for treatment of a male in need of or desiring an improvement of quality of life, sexual domain function or a combination thereof.

Generally speaking, improvement in quality of life (“QOL”) measures refers to an improvement in one or more of happiness, well-being, physical status, emotional status, social status, mental status or cognitive status. Any means used in the field can be used to assess QOL. Typically, QOL of life assessments are based on the reporting of an individual. For example, a number of QOL questionnaires have been developed and are used clinically. One QOL questionnaire used is the SF-36. The SF-36 was used in the specific studies described in the examples. Other examples of QOL instruments include e.g., the WHOQOL-BREF or other as described by Coons et al. Pharmacoeconomics. 2000 January; 17(1):13-35. The ordinary skilled artisan recognizes that other questionnaires or methods for assessing QOL can be used in practice and the invention should not be specifically limited to the use on any particular questionnaire or method. The method of assessing QOL is performed by the individual or subject who is undergoing treatment or alternatively the QOL assessment is made by an external rater (e.g., not the individual or subject undergoing treatment). The QOL questionnaire can be generic like the SF-36 or be a disease specific questionnaire. In some aspects, the method or composition is useful for treating an individual who desires or is in need of improvement in one or more measures of QOL. In some aspects, the method or composition is useful for treating an individual who has had one or more measures of QOL reduced. Any reduction in a measure of QOL is contemplated. Non-limiting examples of reductions of quality in life can be related age, weight, social, physical, emotional, cognitive etc. For example, some individuals report a reduction in QOL measures as they age due to less energy, becoming tired easier, not being able to perform certain activities or perform said certainty activities less efficiently and the such. Another example is individuals who report reduction in QOL due to emotional problems like psychological or psychiatric problems (e.g., disease, condition or disorder), divorce, relationship issues, stress and the such. Yet another example is individuals that report a reduction in QOL measures due to physical problems, like hospitalization, injury, disease, fatigue, etc.

Likewise, a variety of sexual function questionnaires and measures are available to the ordinary skilled artisan including, but not limited to, the MHSQ (see e.g., Rosen et al. Urology. 2004 October; 64(4):777-82), Sexual Health Inventory for Men (SHIM) and Arizona Sexual Experiences Scale (ASEX) (see e.g., Corona et al. International Journal of Impotence Research (2006) 18, 236-250). Sexual function can be assessed by any means including, but not limited to, the subject, the subject's partner, by an external rater, or a combination thereof. Sexual function sometimes is divided into domains like spontaneity, quality of erection, quality of ejaculation, quality of orgasm, sexual pleasure, sexual confidence, erectile function, ejaculation, non-sensuality, avoidance, sexual satisfaction, sexual frequency, sexual communication etc.

Without wishing to be bound by theory, it is believed that the compositions and methods of their use described herein provide levels of testosterone throughout the day that provides unexpected improvements in QOL, sexual domain function or a combination thereof.

In one embodiment, the composition and method described herein provides a serum testosterone Cₘₐₓ to C_{avg,24h} ratio of greater than 1.2 in an individual or group of individuals. In a specific aspect, the serum testosterone Cₘₐₓ to C_{avg,24h} ratio is greater than 1.3, 1.4, 1.5, 1.6, 1.7, 1.8, 1.9, or 2.0 after single dose administration of a composition as described herein at steady state.

In one embodiment, the composition and method described herein provides a serum testosterone C_{avg,12-24h} to C_{avg,12h} ratio of greater than 1.00 in an individual or group of individuals. In a specific aspect, the serum testosterone C_{avg,12-24h} to C_{avg,12h} ratio is greater than 1.05, 1.10, 1.15, 1.20 or 1.25 after 2 doses (e.g., morning dose and evening dose of BID dosing) of a composition as described herein at steady state. For example, the morning dose serum testosterone C_{avg} for 12 hours (C_{avg,12h}) after administration is lower than C_{avg} for 12 hours (C_{avg,12-24h}) after the evening dose. Alternatively, in this embodiment, the serum testosterone levels are generally higher after the evening dose as compared to after the morning dose.

In one embodiment, the method and composition described herein provides safe and efficacious levels of serum testosterone in an individual or group of individuals. Efficacious levels of serum testosterone refers to levels in a normal target range e.g., 250 ng/dL to 1200 ng/dL or within a range defined elsewhere in this paragraph or specification. Safe levels of serum testosterone refers to avoiding high levels of serum testosterone e.g., C_{avg,24h} greater that 1200 ng/dL or 1140 ng/dL and Cₘₐₓ levels less than 3000 ng/dL or 2500 ng/dL or as defined elsewhere in this paragraph or specification.

In one embodiment, the composition and method described herein provides a serum testosterone Cₘₐₓ of less than 1500 ng/dL in greater than or equal to 75%, 80% or 85% of individuals in a group of individuals or a serum testosterone Cₘₐₓ of less than 1500 ng/dL in an individual. In one embodiment, the composition and method described herein provides a serum testosterone Cₘₐₓ as follows: 1800 ng/dL≤Cₘₐₓ≤2500 ng/dL in less than or equal to 10%, 8% or 5% of individuals in a group of individuals. In one embodiment, the composition and method described herein provides a serum testosterone Cₘₐₓ>2500 ng/dL in less than or equal to 5%, 4%, or 3% of individuals in a group of individuals. In one embodiment, the composition and method described herein provides a serum testosterone C_{avg,24h} within 300-1140 ng/dL in greater than 75% of individuals in a group of individuals. In one embodiment, the composition and method described herein provides a serum testosterone C_{avg,24h} within 300-1140 ng/dL in greater than 80%, 85% or 86% of individuals in a group of individuals. In one embodiment, the composition and method described herein provides a serum testosterone C_{avg,24h} within 300-1140 ng/dL in an individual.

As used herein a group of individuals refers to 2 or more individuals, preferably 10 or more individuals. In some aspects, a group of individuals refers to more than: 15, 20, 25, 30, 40, 50, 60, 70, 80, 90, or 100 individuals.

In some embodiments, the methods and compositions are employed for more than 1 week to achieve the improvement in QOL or sexual function. In specific aspects, the methods and compositions are employed for 2, 3, 4, 5, 6, 7, 8, 9, 10, 11, or 12 or more weeks to achieve the improvement in QOL or sexual function. In other specific aspects, the methods and compositions are employed for 2, 3, 4, 5, 6, 7, 8, 9, 10, 11, or 12 or more months to achieve the improvement in QOL or sexual function.

The improvements described herein for QOL and sexual function can be in reference to baseline. Baseline can refer to e.g., prior to commencing the oral testosterone therapy described herein), in reference to the subject when testosterone deficient (e.g., prior to commencing the oral testosterone therapy described herein and/or after wash-out of any prior testosterone replacement therapy), in reference to a previous testosterone therapy (e.g., transdermal/transmucosal, injectable, nasal, or other oral) or a combination thereof. In some aspects, the improvement is assessed by comparisons with a group of population of subjects.

In one aspect, the improvement is statistically significant.

In specific embodiments, provided herein are the following:
1. A method for replacement therapy in a male for having a condition or a symptom associated with a deficiency or absence of endogenous testosterone, said method comprising: orally administering to a male having a condition or a symptom associated with a deficiency or absence of endogenous testosterone, with a meal, a pharmaceutical composition comprising from about 50 mg to about 300 mg of testosterone undecanoate and a pharmaceutically acceptable carrier said method providing an improvement in one or more quality of life measures or sexual domain function measures.
2. The method of claim 1 said improvement in quality of life or sexual domain function is measurable by a questionnaire.
3. The method of 1 said quality of life measurement is measurable by the SF-36 questionnaire.
4. The method of 1 said quality of life measurement is measurable by the SF-36 questionnaire and is physical functioning, role physical, vitality, social functioning, mental health, physical component, mental component or a combination thereof.
5. The method of 1 said sexual domain function is libido, erection, ejaculation, orgasm, satisfaction or a combination thereof.
6. The method of 1 said sexual domain function is overall level of sexual desire, pleasure with partner, pleasure without partner, positive mood, less negative mood, sexual activity, full erections, maintained erections or a combination thereof.
7. The method as in 1, said pharmaceutical composition is a unit dosage form having about 75 mg, about 112.5 mg, about 150 mg, about 225 mg, or about 300 mg of testosterone undecanoate.
8. The method as in 1 said pharmaceutical acceptable carrier selected to provide bioequivalent amounts of serum testosterone levels to said male for meals containing low, standard fat and high fat.
9. The method as in 1 said method providing a serum testosterone C_{avg,24h} in the range of 300 ng/dL to 1140 ng/dL.
10. The method as in 1, said administering is twice-a-day.
11. The method as in 1, said method comprising administering: (a) from 285 mg to about 625 mg of testosterone undecanoate per day; (b) about 300 mg per day; (c) about 450 mg per day; or (d) about 600 mg per day.
12. The method as in 1, said composition comprising a lipophilic additive.
13. The method as in 1, said composition comprising a hydrophilic additive.
14. The method as in 1, said pharmaceutical composition being pharmaceutically equivalent, bioequivalent or both to an oral pharmaceutical composition (1) having (a) about 75 mg or about 112.5 mg of testosterone undecanoate at about 15% loading and (b) about 63% Maisine 35-1, about 16% Cremophor RH 40 and about 6% PEG 8000 or (2) having (a) about 75 mg, about 112.5 mg, about 150 mg, about 225 mg or about 300 mg of testosterone undecanoate at about 15% loading and (b) about 63% Maisine 35-1, about 16% Cremophor RH 40 and about 6% PEG 8000.
15. The method as in 1, said method comprising administering the pharmaceutical composition as 2, 3, 4, 5, 6, 7, or 8 unit dosage forms per day.
16. A method for replacement therapy in a male for a condition or symptom associated with a deficiency or absence of endogenous testosterone, said method comprising: orally administering to a male having a condition or symptom associated with a deficiency or absence of endogenous testosterone, a pharmaceutical composition comprising from about 50 mg to about 300 mg of testosterone undecanoate and a pharmaceutically acceptable carrier said method providing an improvement in one or more quality of life measures.
17. The method of 16 said improvement in quality of life is measurable by a questionnaire.
18. The method of 16 said quality of life measurement is measurable by the SF-36 questionnaire.
19. The method of 16 said quality of life measurement is measurable by the SF-36 questionnaire and is physical functioning, role physical, vitality, social functioning, mental health, physical component, mental component or a combination thereof.
20. The method as in 16, said pharmaceutical composition being pharmaceutically equivalent, bioequivalent or both to an oral pharmaceutical composition (1) having (a) about 75 mg, about 112.5 mg, about 150 mg, about 225 mg or about 300 mg of testosterone undecanoate at about 15% loading and (b) about 63% Maisine 35-1, about 16% Cremophor RH 40 and about 6% PEG 8000 or (2) having (a) about 75 mg or about 112.5 mg of testosterone undecanoate at about 15% loading and (b) about 63% Maisine 35-1, about 16% Cremophor RH 40 and about 6% PEG 8000.
21. The method as in 16, said method providing a serum testosterone C_{avg,24h} in the range of 300 ng/dL to 1140 ng/dL.
22. The method as in 16, said method comprising administering from about 285 mg to about 625 mg of testosterone undecanoate per day.
23. The method of claim 16 said one or more quality of life measures is physical role, vitality, social functioning, mental health, mental component summary or a combination thereof.
24. The method as in 16, said administering is twice-a-day.
25. A method for replacement therapy in a male for a condition or symptom associated with a deficiency or absence of endogenous testosterone, said method comprising: orally administering to a male having a condition or symptom associated with a deficiency or absence of endogenous testosterone, a pharmaceutical composition comprising from about 50 mg to about 300 mg of testosterone undecanoate and a pharmaceutically acceptable carrier said method providing an improvement in sexual domain function.
26. The method of 25 said sexual domain function being libido, erection, ejaculation, orgasm, satisfaction or a combination thereof.
27. The method of 25 said sexual domain function being overall level of sexual desire, pleasure with partner, pleasure without partner, positive mood, less negative mood, sexual activity, full erections, maintained erections or a combination thereof.
28. A pharmaceutical composition for oral administration comprising testosterone undecanoate and a pharmaceutical acceptable carrier said pharmaceutical composition providing bioequivalent amounts of serum testosterone undecanoate, testosterone, dihydrotestosterone, dihydrotestosterone undecanoate or a combination thereof to an oral pharmaceutical composition having about 75 mg or about 112.5 mg of testosterone undecanoate at 15% loading, 63% Maisine 35-1, 16% Cremophor RH 40 and 6% PEG 8000 when administered orally twice a day to a male provides an improvement in one or more measures of sexual domain function, quality of life or a combination thereof.
29. The pharmaceutical composition as in 28 comprising a monoglyceride, a diglyceride or a combination thereof in amount of greater than about 10 wt % and has less than about 50 wt % triglyceride.
30. A pharmaceutical composition for oral administration that is hypogonadal male serum testosterone bioequivalent to a formulation comprising 15% testosterone undecanoate, about 63% Maisine 35-1, about 16% Cremophor RH 40, and about 6% polyethylene glycol 8000 with the proviso that the formulation does not comprises about 15% testosterone undecanoate, about 63% Maisine 35-1, about 16% Cremophor RH 40, and about 6% polyethylene glycol 8000 and when administered orally twice a day with a meal to a male provides an improvement in one or more measures of sexual domain function, quality of life or a combination thereof.
31. The pharmaceutical composition as in 30, said pharmaceutical composition being pharmaceutically equivalent to an oral pharmaceutical composition having about 75 mg, about 112.5 mg, about 150 mg, about 225 mg or about 300 mg of testosterone undecanoate at 15% loading, 63% Maisine 35-1, 16% Cremophor RH 40 and 6% PEG 8000.
32. The pharmaceutical composition as in 30 comprising a monoglyceride, a diglyceride or a combination thereof in amount of greater than 10 wt % and has less than 50 wt % triglyceride.
33. A pharmaceutical composition for oral administration for replacement therapy in a male for a condition or symptom associated with a deficiency or absence of endogenous testosterone comprising testosterone undecanoate and a pharmaceutical acceptable carrier said pharmaceutical composition indicated to be taken (1) “WITH A MEAL”, (2) “WITH MEAL, BUT NOT ON EMPTY STOMACH”, (3) “WITH FAT CONTAINING FOOD” not specifying fat content, (4) “WITH MEAL, BUT NOT LOW FAT”, (5) “WITH MEAL, BUT NOT HIGH FAT”, (6) “WITH STANDARD OR NORMAL MEAL” or (7) “WITH MEAL. BUT NOT HIGH FAT” when administered orally twice a day with a meal to a male provides an improvement in one or more measures of sexual domain function, quality of life or a combination thereof.
34. The pharmaceutical composition as in 33 said pharmaceutical composition indicated to be taken “WITH A MEAL”.
35. The pharmaceutical composition as in 33 said pharmaceutical composition indicated to be taken “WITH MEAL. BUT NOT ON EMPTY STOMACH”.
36. The pharmaceutical composition as in 33 said pharmaceutical composition indicated to be taken “WITH FAT CONTAINING FOOD” not specifying fat content.
37. The pharmaceutical composition as in 33 said pharmaceutical composition indicated to be taken “WITH MEAL, BUT NOT LOW FAT”.
38. The pharmaceutical composition as in 33 said pharmaceutical composition indicated to be taken “WITH MEAL, BUT NOT HIGH FAT”.
39. The pharmaceutical composition as in 33 said pharmaceutical composition indicated to be taken “WITH STANDARD OR NORMAL MEAL”
40. The pharmaceutical composition as in 33 said pharmaceutical composition indicated to be taken “WITH MEAL. BUT NOT HIGH FAT”.
41. The method of any one of 1-27 further comprising a dose titration of testosterone undecanoate.
42. The method of any one 1-27 or 41 with a pharmaceutical composition as in any one of 28-40 to the extent the methods and compositions are not inconsistent with one another.

Before the present testosterone undecanoate compositions, oral dosage capsules and related methods of use are disclosed and described in more detail and variations, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

### Definitions

As used herein, “pharmaceutically equivalent”, refers to a composition or unit dosage form drug product if they meet three criteria: they contain the same active ingredient(s); they are of the same dosage form and route of administration; they are identical in strength or concentration. Typically pharmaceutical equivalent drug products may differ in characteristics such as shape, release mechanism, labeling (to some extent), scoring and excipients (including colors, flavors, preservatives) although this list in not-limiting.

As used herein, “bioequivalent”, refers to the absence of a significant difference in the rate and extent to which the active ingredient or active moiety in pharmaceutical equivalents or pharmaceutical alternatives becomes available at the site of drug action when administered at the same dose under similar conditions in an appropriately designed study. Two products are bioequivalent if the 90% CI (Confidence Interval) of the relative mean Cₘₐₓ, AUC₍₀₋ₜ₎ and AUC_{(0-∞)} of the test (e.g., generic formulation) to reference (e.g., innovator brand formulation) should be within 70% to 143% and preferably 80.00% to 125.00% under particular conditions e.g., fed, fasted, particular fat content in meals (e.g., low, standard high). Unless otherwise specified, bioequivalence can be for serum testosterone undecanoate, serum testosterone, serum dihydrotestosterone or serum dihydrotestosterone undecanoate. Typically bioequivalence is determined with a group of subjects.

As used herein, the term “treatment,” when used in conjunction with the administration of pharmaceutical compositions and oral dosage capsules containing testosterone undecanoate, refers to the administration of the oral dosage capsules and pharmaceutically acceptable composition to subjects who are either asymptomatic or symptomatic. In other words, “treatment” can both be to reduce or eliminate symptoms associated with a condition present in a subject, or it can be prophylactic treatment, i.e. to prevent the occurrence of the symptoms in a subject. Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Furthermore, the term “dosage form” can include one or more formulation(s) or composition(s) provided in a format for administration to a subject. When any of the above terms is modified by the term “oral” such terms refer to compositions, formulations, or dosage forms formulated and intended for oral administration to subjects.

As used herein, the term “fatty acid” refers to unionized carboxylic acids with a long aliphatic tail (chain), either saturated or unsaturated, conjugated or non-conjugated. Typically, the fatty acid is a C₈ to C₂₂ fatty acid.

Unless otherwise specified, the term C₈ to C₂₂ fatty acid glycerides refers to a mixture of mono-, di-, and/or tri-glycerol esters of medium to long chain (C₈ to C₂₂) fatty acids.

As used herein, the term “solidifying agent” or “solidifying additive” are used interchangeably and refer to a pharmaceutically acceptable additive that is in a solid physical state at 20° C. Similarly, a “solid lipophilic additive” refers to a lipophilic compound or component that is in a solid physical state at 20° C., and/or renders the composition or dosage form non-liquid, such as solid or semi-solid.

As used herein, the term “lipophilic.” refers to compounds that are not freely soluble in water; and the term “lipophilic surfactant” refers to surfactants that have HLB values of about 10 or less. Conversely, the term “hydrophilic” refers to compounds that are soluble in water, and term “hydrophilic surfactant” refers to surfactants that have HLB values of more than about 10.

As used herein, the term “ionizable fatty acid” refers to a fatty acid compound that changes its HLB as a function of pH. For example oleic acid is predominantly lipophilic at lower pH values (such as those found in the stomach) but becomes a predominantly hydrophilic at higher pH values (such as those found in the intestine).

As used herein, “subject” refers to a mammal that may benefit from the administration of a drug composition or method of this invention. Examples of subjects include humans. In one aspect, the subject can be a human male. In another embodiment, the subject can be a hypogonadal male. In one aspect, the subject is a male in need of replacement therapy for conditions associated with a deficiency or absence of endogenous testosterone. As used herein, the testosterone deficiency or hypogonadism in a male human subject (hypogonadal male) refers to a condition wherein the average baseline plasma testosterone concentration (T-C_{avg-B}) is about 300 ng/dL or less. However in some instances, testosterone deficiency or hypogonadism in a male human subject refers to a condition wherein the average baseline plasma testosterone concentration is about 400 ng/dL or less.

As used herein, “group” or “group of subjects” refers to a collection of at least 12 human male subjects who receive and respond to exogenous oral administration of the compositions disclosed herein, namely testosterone undecanoate-containing compositions. In one aspect, the group can include at least 100 or at least 300 male subjects. In another aspect, the group can include at least 1000 male subjects. In another embodiment, the subjects can be hypogonadal subjects.

The term “oral administration” represents any method of administration in which an active agent can be administered by swallowing, chewing, or sucking of the dosage form. The composition of the current inventions can be admixed with food or drink prior to being orally consumed.

As used herein, a “dosing regimen” or “regimen” such as an “initial dosing regimen” or a “maintenance dosing regimen” refers to how, when, how much, and for how long a dose of the compositions of the present invention can be administered to a subject. For example, an initial dosing regimen for a hypogonadal male subject may provide for a total daily dose of 450 mg administered in two divided doses at about 12 hours apart (e.g., once with breakfast and once with dinner) with meals (e.g., having about 10 g to 55 g of fat content, or any other appropriate meal) repeated daily for at least one week, two weeks or 30 days.

As used herein, “daily dose” refers to the amount of active agent (e.g., testosterone undecanoate) administered to a subject over a 24 hour period of time. The daily dose can be administered two or more administrations during the 24 hour period. In one embodiment, the daily dose provides for two administrations in a 24 hour period. With this in mind, an “initial dose” or initial daily dose” refers to a dose administered during the initial regimen or period of a dosing regimen. An initial dose includes both the very first dose during the initial regimen as well as the subsequent doses during the same initial regimen. Similarly, a “maintenance dose” or “maintenance daily dose” refers to a dose administered during a maintenance regimen of a dosing regimen. It is worth noting that the maintenance dose follows a dose titration based on the serum testosterone determination on a titration node day (after a single dose of the initial daily dose at steady state), however the maintenance dose does not need to be of a different quantity as the initial dose or the previous maintenance dose (in the case of multiple titrations).

As used herein, the term “solidifying agent” or “solidifying additive” are used interchangeably and refer to a pharmaceutically acceptable additive that is in a solid physical state at 20° C. Similarly, a “solid lipophilic additive” refers to a lipophilic compound or component that is in a solid physical state at 20° C., and/or renders the composition or dosage form non-liquid, such as solid or semi-solid.

As used herein, “non-liquid” when used to refer to the state of a composition disclosed herein refers to the physical state of the composition as being a semi-solid or solid.

As used herein, “solid” and “semi-solid” refers to the physical state of a composition that supports its own weight at standard temperature and pressure, and has adequate viscosity or structure to not freely flow. Semi-solid materials may conform to the shape of a container under applied pressure.

As used herein, “titration” or “dose titration” or “dose adjustment” are used interchangeably and refer to an increase or decrease of the total daily dose of testosterone undecanoate administered to a subject, typically based on the response of the subject to the exogenous administered testosterone undecanoate. The dose can be increased or decreased based on the measurement of serum testosterone concentration after a steady state has been achieved.

As used herein, “steady state” refers to the achievement of a stable response in serum total testosterone levels to exogenously administered testosterone undecanoate, typically achieved after at least 15 days following the start of a dosing regimen.

As used herein, “initial daily dose” (IDD) or “Daily dose of the initial regimen” is a dose of testosterone undecanoate administered daily to a subject in need of testosterone therapy. The initial daily dose may be administered in two or more intervals over a 24 hour period, e.g., twice-a-day. Similarly, “maintenance daily dose” or “daily dose of the maintenance regiment” is a dose of testosterone undecanoate administered daily to a subject in need of testosterone therapy as determined based on measurement of the titration node day titration metric and is the daily dose going forward within a few days of measurement unless a dose change is needed based on a another titration node day measurements. During a maintenance regime there may be two or more daily doses administered which at some point during the regime would be considered to be the maintenance daily dose.

As used herein, “titration node” or “titration node day” are used interchangeably and refer to a day on which a serum sample is drawn from a subject for measurement of the serum testosterone concentrations in order to determine whether a testosterone undecanoate dose titration is necessary and what the titration type might need to be. The measured serum testosterone levels may also be used to determine dose a titration metric to be utilized in deciding dose titration needs for an individual subject. As dosing regimens can include one or more titration node day the term may refer to a first titration node during a dosing regimen (e.g. between the initial dosing regimen and the maintenance dosing regimen) or it can refer to a subsequent titration node day between a maintenance dosing regimen and a subsequent maintenance dosing regimen.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision.

The determination of an effective amount is well within the ordinary skill in the art of pharmaceutical sciences and medicine. See, for example. Meiner and Tonascia, “Clinical Trials: Design, Conduct, and Analysis.” Monographs in Epidemiology and Biostatistics, Vol. 8 (1986), incorporated herein by reference.

The terms “plasma testosterone concentration.” “testosterone concentration in the blood.” and “serum testosterone concentration” are used interchangeably and refer to the “total” testosterone concentration which is the sum of the bioavailable testosterone including free and protein-bound testosterone concentrations. As with any bio-analytical measure, for increased consistency the method employed to measure initial serum testosterone levels should be consistent with the method used to monitor and re-measure serum testosterone levels during clinical testing and testosterone therapy for a subject. Likewise, serum or plasma (used interchangeably) testosterone undecanoate and the metabolites dihydrotestosterone and dihydrotestosterone undecanoate, can be determined by the ordinary skilled artisan. “Free testosterone” refers to testosterone that is not bound to SHBG or other proteins like albumin and is readily determined by an ordinary skilled artisan. Free testosterone levels in adult males are typical about 1%-2% of total testosterone levels.

As used herein “SHBG” refers to sex hormone binding globulin.

As used herein, of the average serum testosterone concentration can be determined using methods and practices known in the art. For example, the average baseline plasma testosterone concentration of a human male is the arithmetic mean of the total plasma testosterone concentrations determined on at least two consecutive time points that are reasonably spaced from each other, for example from about 1 hour to about 168 hours apart. In a particular case, the plasma testosterone concentration can be determined on at least two consecutive times that are about 12 hours to about 48 hours apart. In another particular method, the plasma testosterone concentration of the human male can be determined at a time between about 5 o'clock and about 11 o'clock in the morning. Further, the plasma testosterone concentration can be the determined by standard analytical procedures and methods available in the art, such as for example, automated or manual immunoassay methods, liquid chromatography or liquid chromatography-tandem mass spectrometry (LC-MSMS) etc.

As used herein, the term AUC₀₋ₜ is the area under the curve of a plasma-versus-time graph determined for the analyte from the time 0 to time “t”.

As used herein, the term “C_{avg},” “Cₐᵥₑ,” or “C-average” are used interchangeably, and is determined as the AUC₀₋ₜ or the mean AUC divided by the time period (t). For example C_{avg-8h} is the average plasma concentration over a period of 8 hours post-dosing determined by dividing the AUC₀₋₈ value by 8. Similarly, C_{avg-12h} is the average plasma concentration over a period of 12 hours post-dosing determined by dividing the AUC₀₋₁₂ value by 12; C_{avg-24h} is the average plasma concentration over a period of 24 hours post-dosing determined by dividing the AUC₀₋₂₄ value by 24, and so on. Unless otherwise stated, all Cₐᵥₑ values are considered to be Cₐᵥₑ₋₂₄ₕ.

As used herein “Cₘₐₓ” refers to the maximum measured serum concentration of the administered drug or a metabolite (e.g., testosterone) after single dose administration.

As used herein, “free of” or “substantially free of” of a particular compound or compositions refers to the absence of any separately added portion of the referenced compound or composition. Free of or substantially free of can include the presence of 1 wt % or less (based on total composition weight) of the referenced compound which may be present as a component or impurity of one or more of the ingredients.

As used herein, the term “TU” refers to testosterone undecanoate.

As used herein, “with a meal” general means within an hour of a meal (e.g., plus/minus an hour or preferably within 30 minutes). More preferably. “with a meal” means within 30 minutes of a meal e.g., within 30 minutes after the subject has eaten a meal. Even more preferably, “with a meal” refers to about 30 minutes after the subject has eaten a meal. The terms “meal” and “food” can be used interchangeably.

As used herein, the term “about” is used to provide flexibility to a numerical range endpoint by providing that a given value may be “a little above” or “a little below” the endpoint. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Described herein are formulations and methods of using those formulations for testosterone replacement therapy. The formulations and methods can be used for oral administration for an individual or subject seeking an improvement in a QOL measure or sexual function. The formulations and methods can be used for oral administration for an individual or subject having one or more baseline serum testosterone measurements of less than 350, 325, 300, 275, 250, 200, 175, 150, 125, 100, 75 or 50 ng/dL. The formulations and methods can be used for oral administration for an individual or subject having one or more baseline serum testosterone measurements of from 400-600, 300-500, 300-400, 300-350, 275-325, 250-300, 225-275, 200-250, 150-200, 125-175, 100-150, 75-125, 50-100, 25-75 or 1-50 ng/dL. In some aspects, the formulations described herein are for oral administration for replacement therapy in a male for conditions associated with a deficiency or absence of endogenous testosterone (e.g., a hypogonadal male or a male experiencing a symptom of testosterone deficiency). The formulations can be any formulation having a testosterone ester e.g., testosterone undecanoate and one or more carriers. In one aspect, the carriers are selected such that the formulation (containing TU), when administered orally for replacement therapy in a male for conditions associated with a deficiency or absence of endogenous testosterone, produces bioequivalent serum testosterone levels when administered with low, standard and high fat meals. In some aspects, the carriers and amounts thereof are selected such that the formulation provides bioequivalent levels of serum testosterone when administered under high fat versus low fat meals, standard meals versus low fat, standard versus high fat, or low, standard and high fat meals. In one aspect, the composition or oral dosage form can be administered with a meal, such as a meal that provides about 200 calories to about 1000 calories of energy. In another aspect, the composition or oral dosage form can be administered with a meal that provides about 5%, 10%, 15%, 20%, 25%, 30%, 35%, 40%, 45%, 50%, 55%, 60%, 70%, 80%, 90% or 100% of the calories from the fat (or within a range derived from any combination of these values). In yet another aspect, the composition or oral dosage form can be administered with a meal that provides about 5%, 10%, 15%, 20%, 25%, 30%, 35%, 40%, 45%, or 50% of the calories from the fat (or within a range derived from any combination of these values). In another aspect, the composition or oral dosage form can be administered with a high-fat, high calorie meal. In another aspect, the composition or oral dosage form can be administered with a standard meal that provides about 500 calories to about 1000 calories of energy. The compositional make-up of the meals that are administered can vary depending on the tastes and dietary needs of a subject. However, in some situations it may be beneficial to administer the compositions and oral dosage forms with meals that provide no fat to about 100 g of fat. In one aspect, the meal can provide about 10 g to about 50 g of fat. In yet a further aspect, the meal can provide 15 g to about 35 g of fat. In yet a further aspect, the meal can provide about 1, 2, 3, 4, 5, 6, 7, 8, 9, 10, 11, 12, 13, 14, 15, 16, 17, 18, 19.20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51.52, 53, 54, 55, 56, 57, 58, 59, 60 grams of fat (or within a range derived from any combination of these values). According to this embodiment, the method and composition is capable of providing an improvement in one or more quality of life measures or sexual domain function. In one aspect, the improvement in quality of life or sexual domain function is measurable by a questionnaire. In a specific aspect, the quality of life measurement is measurable by the SF-36 questionnaire. In another specific aspect, the quality of life measurement is measurable by the SF-36 questionnaire and is physical functioning, role physical, vitality, social functioning, mental health, physical component, mental component or combination thereof. In again another aspect, the improvement is sexual domain function is libido, erection, ejaculation, orgasm, satisfaction or a combination thereof. In still another aspect, the improvement in sexual domain function is overall level of sexual desire, pleasure with partner, pleasure without partner, positive mood, less negative mood, sexual activity, full erections, maintained erections or a combination thereof. The improvements referred to in this paragraph can be in reference to baseline.

In one embodiment, a testosterone replacement therapy for twice daily oral dosing is provided comprising: (a) 2 (or 3) different dose strength oral dosage forms having different amounts of testosterone undecanoate; (b) 3 dosing regimens providing for 3 different daily doses of testosterone undecanoate; (c) both (a) and (b); or (d) a pharmaceutically equivalent version thereof. In one aspect, the testosterone replacement therapy provides steady state serum levels of testosterone (C_{avg}) to a male having testosterone deficiency or in need of said therapy in the range of about 300 ng/dL to about 1140 ng/dL. In one aspect, the testosterone replacement therapy provides steady state serum levels of testosterone (C_{avg}) to a male having testosterone deficiency or in need of said therapy in the range of about 400 ng/dL or 435 ng/dL to about 1140 ng/dL. In one aspect, the testosterone replacement therapy provides single dose Cₘₐₓ levels of serum testosterone at steady state to a population of males having testosterone deficiency, or in need of said therapy, of less than 2500 ng/dL in at least 95% of the population of males, less than 1500 ng/dL in at least 85% of the population of males; or a serum testosterone Cₘₐₓ of about 1800 ng/dL to about 2500 ng/dL in 10% or less of the population of males having testosterone deficiency. In one aspect, the testosterone replacement therapy has one of the dosage forms having from about 140 to 160 mg testosterone undecanoate and the other dosage form has from about 215 mg to about 250 mg testosterone undecanoate. In one aspect, the testosterone replacement therapy can include a third dose strength that has from about 280 mg to about 320 mg of testosterone undecanoate. In one specific aspect, the third dose strength has about 300 mg testosterone undecanoate. In one aspect, the testosterone replacement therapy has one of the dosage forms having from about 145 to 155 mg testosterone undecanoate and the other dosage form has from about 220 mg to about 230 mg testosterone undecanoate. In one aspect, the testosterone replacement therapy has one the of dosage forms having about 150 mg testosterone undecanoate and the other dosage form has about 225 mg testosterone undecanoate. In one aspect, the testosterone replacement therapy has one the of dosage forms having from about 60 to 90 mg testosterone undecanoate and the other dosage form has from about 100 mg to about 130 mg testosterone undecanoate. In one aspect, the testosterone replacement therapy has one of the dosage forms having from about 70 to 80 mg testosterone undecanoate and the other dosage form has from about 107 mg to about 118 mg testosterone undecanoate. In one aspect, the testosterone replacement therapy has one of dosage forms having about 75 mg testosterone undecanoate and the other dosage form has about 112.5 mg testosterone. In one aspect, the testosterone replacement therapy has 3 dosing regimens providing for 3 different daily doses of testosterone undecanoate provide for a first daily dose of about 275 mg to about 325 mg of testosterone undecanoate, a second daily dose of from about 425 mg to about 490 mg of testosterone undecanoate and a third daily dose of about 575 mg to about 625 mg of testosterone undecanoate. In one aspect, the testosterone replacement therapy provides for 3 different daily doses of testosterone undecanoate-providing for a first daily dose of about 300 mg of testosterone undecanoate, a second daily dose of about 450 testosterone undecanoate and a third daily dose of about 600 mg of testosterone undecanoate. In one aspect, the testosterone replacement therapy comprises: 2 dosage forms, one having about 75 mg of testosterone undecanoate and the other dosage form having about 112.5 testosterone undecanoate; 3 dosing regimens, a first dosing regimen comprising administration of two dosage forms twice a day each dosage form having about 75 mg testosterone undecanoate, a second dosing regimen comprising administration of two dosage forms twice a day each dosage form having about 112.5 mg testosterone undecanoate, and a third dosing regimen comprising administration of four dosage forms twice a day each dosage form having about 75 mg testosterone undecanoate or a pharmaceutically equivalent version thereof. In one aspect, the testosterone replacement therapy comprises: 2 dosage forms, one having about 150 mg of testosterone undecanoate and the other dosage form having about 225 testosterone undecanoate; 3 dosing regimens, a first dosing regimen comprising administration of one dosage form twice a day each dosage form having about 225 mg testosterone undecanoate, a second dosing regimen comprising administration of two dosage forms twice a day each dosage form having about 150 mg testosterone undecanoate, and a third dosing regimen comprising administration of two dosage forms twice a day each dosage form having about 150 mg testosterone undecanoate or a pharmaceutically equivalent version thereof. As described herein, administration refers to administration to a subject in need of testosterone replacement e.g., a hypogonadal male or a male having low testosterone levels or a symptom thereof. According to one aspect of this embodiment, the dosage form can be a capsule with the fill containing 112.5 mg TU at about 15% loading, 63% Maisine 35-1, 16% Cremophor RH 40 and 6% PEG 8000) or a pharmaceutically equivalent formulation thereof, a bioequivalent formulation thereof or a combination thereof. According to this embodiment, the method and composition is capable of providing an improvement in one or more quality of life measures or sexual domain function. In one aspect, the improvement in quality of life or sexual domain function is measurable by a questionnaire. In a specific aspect, the quality of life measurement is measurable by the SF-36 questionnaire. In another specific aspect, the quality of life measurement is measurable by the SF-36 questionnaire and is physical functioning, role physical, vitality, social functioning, mental health, physical component, mental component or combination thereof. In again another aspect, the improvement is sexual domain function is libido, erection, ejaculation, orgasm, satisfaction or a combination thereof. In still another aspect, the improvement in sexual domain function is overall level of sexual desire, pleasure with partner, pleasure without partner, positive mood, less negative mood, sexual activity, full erections, maintained erections or a combination thereof. The improvements referred to in this paragraph can be in reference to baseline.

As described herein an oral TRT (testosterone replacement therapy) is provided. The TRT has three different daily doses which commence with an initial dosing regimen having a specific daily dose of testosterone undecanoate that last for a period of time e.g., at least 1, 2, 3, 4, 5, 6, 7, 8, 9, 10, 11, 12, 13, 14, 15, or 16 weeks. After this period of time on the initial dosing regimen, a dose titration measurement or assessment is made. The purpose of the dose titration assessment or measurement is to determine if the daily dose should remain the same as daily dose of the initial regimen or whether the daily dose should be increased or decreased. The dose titration measurement or assessment is made by determining serum testosterone concentrations within a specific amount of time (e.g., window of time) after administration of a single dose of the initial regimen at steady state. Three options are possible based on the result of this measurement or assessment. A level of serum testosterone that is too high will result in a decrease in the total daily dose of testosterone undecanoate, a level of serum testosterone that is too low will result in an increase in the daily dose of testosterone undecanoate and intermediate levels of serum testosterone will result in no change of the daily dose of testosterone undecanoate. According to this TRT, a subject or patient e.g., hypogonadal male starts on an initial dose or initial dosing regimen that provides a specific amount of testosterone undecanoate per day for an initial period of time (e.g., greater than one, two, or three weeks) that is to be administered with food. This initial daily dose is in the range of about 430 mg to 490 mg TU per day (or 435 mg to 465 mg TU per day, 440 mg to 460 mg TU per day, 445 mg to 455 mg TU per day, or about 450 mg per day). After the initial period of time on the initial daily dose (e.g., more than 1, 2, 3, or 4 weeks), a dose titration measurement or assessment is made. The dose titration measurement is made by determining serum testosterone levels at a specific time (e.g., within 1 to 12 hours, 1 to 11 hours, 1 to 10 hours, 1 to 9 hours, 1 to 8 hours, 1 to 7 hours, 1 to 6 hours, 1 to 5 hours, 1 to 4 hours, 2 to 10 hours, 2 to 9 hours, 2 to 8 hours, 2 to 7 hours, 2 to 6 hours, 2 to 5 hours, 2 to 4 hours, 1 to 3 hours, 2 to 3 hours, 3 to 4 hours, 4 to 5 hours, 3 to 5 hours, 4 to 6 hours, 3 to 6 hours, 3 to 8 hours, or 4 to 6 hours; or at about 1, 2, 3, 4, 5, 6, 7, 8, 9, 10, 11, or 12 hours±0.5, 1, 1.5, or 2 hours) after a single dose of the initial regimen when the patient is at steady state. Depending of the serum testosterone level obtained from the dose titration measurement, the patient can receive a maintenance regimen that has the same daily dose as the initial regimen or the daily dose is increased or decreased. Typically, the patient or subject is then maintained on the maintenance regimen, although one or more additional dose titration measurements or dose titration can be made. The therapy described herein is typically administered as a twice a day therapy with a meal, so a 300 mg TU dose is administered as 150 mg with a meal twice a day; a 450 mg dose is administered as 225 mg with a meal twice a day; and a 600 mg dose is administered as 300 mg with a meal twice a day. According to this embodiment, the method and composition is capable of providing an improvement in one or more quality of life measures or sexual domain function. In one aspect, the improvement in quality of life or sexual domain function is measurable by a questionnaire. In a specific aspect, the quality of life measurement is measurable by the SF-36 questionnaire. In another specific aspect, the quality of life measurement is measurable by the SF-36 questionnaire and is physical functioning, role physical, vitality, social functioning, mental health, physical component, mental component or combination thereof. In again another aspect, the improvement is sexual domain function is libido, erection, ejaculation, orgasm, satisfaction or a combination thereof. In still another aspect, the improvement in sexual domain function is overall level of sexual desire, pleasure with partner, pleasure without partner, positive mood, less negative mood, sexual activity, full erections, maintained erections or a combination thereof. The improvements referred to in this paragraph can be in reference to baseline.

The dose titration of the TRT described herein was found to be surprisingly robust and beneficial for patients receiving the therapy. In a specific aspect, the up or down titration are at about 70 to 80 mg TU per dose (e.g., 75 mg TU) or about 140 to 160 mg (e.g., 150 mg TU) per day TU. Typically, the doses of TU are administered with a meal. Thus, the methods described here can include an initial daily dose followed by a dose titration. The dose titration is used to determine the maintenance daily dose which is within plus/minus 75%, 50%, 40% or 35% of the initial daily dose.

In one embodiment, a therapy for treating a male having a baseline serum testosterone level of 300 ng/dL or less is provided said therapy comprising: (a) 2 oral dosage forms having different amounts of testosterone undecanoate; (b) 3 dosing regimens providing different daily doses of testosterone undecanoate; (c) both (a) and (b); or (d) a pharmaceutically equivalent version thereof. In one aspect, the higher and lower daily doses are within about 40% of the intermediate dose. In one aspect, the therapy provides single dose Cₘₐₓ levels of serum testosterone at steady state levels to a population of males having testosterone deficiency, or in need of said therapy, of (a) less than 2500 ng/dL in at least 95% of the population of males; (b) less than 1500 ng/dL in at least 85% of the population of males; a serum testosterone Cₘₐₓ of about 1800 ng/dL to about 2500 ng/dL in 10% or less of the subjects in the group; or a combination thereof. In one aspect, a patient receiving the therapy has a dose titration assessment. In one aspect, the dose titration assessment comprises determining a value of serum testosterone at from about two to eight hours after receiving a dose of testosterone undecanoate. In one aspect, a patient having (a) a low serum testosterone level at two to eight hours after receiving a single dose of testosterone undecanoate at steady state receives a higher dose of testosterone undecanoate; (b) a high serum testosterone level at two to eight hours after receiving a single dose of testosterone undecanoate at steady state receives a lower dose of testosterone undecanoate: (c) an intermediate serum testosterone level after receiving a single dose of testosterone undecanoate at steady state an intermediate dose of testosterone undecanoate; or (d) a combination thereof. In one aspect, the dose titration assessment comprises determining the serum testosterone level during a window period within two to eight hours after receiving a dose of testosterone undecanoate. Low serum testosterone can be defined as less than 500, 450, 400, 350, 300, 250 or 200 ng/dL. High serum testosterone can be defined as greater than 500, 550, 600, 650, 700, 750, 800 or 850 ng/dL. Intermediate serum testosterone can be defined as a range of values defined by low and high serum testosterone levels in the previous two sentences. According to this embodiment, the method and composition is capable of providing an improvement in one or more quality of life measures or sexual domain function. In one aspect, the improvement in quality of life or sexual domain function is measurable by a questionnaire. In a specific aspect, the quality of life measurement is measurable by the SF-36 questionnaire. In another specific aspect, the quality of life measurement is measurable by the SF-36 questionnaire and is physical functioning, role physical, vitality, social functioning, mental health, physical component, mental component or combination thereof. In again another aspect, the improvement is sexual domain function is libido, erection, ejaculation, orgasm, satisfaction or a combination thereof. In still another aspect, the improvement in sexual domain function is overall level of sexual desire, pleasure with partner, pleasure without partner, positive mood, less negative mood, sexual activity, full erections, maintained erections or a combination thereof. The improvements referred to in this paragraph can be in reference to baseline.

In one embodiment, a method for replacement therapy in a male for having a condition or a symptom associated with a deficiency or absence of endogenous testosterone is provided, said method comprising: orally administering to a male having a condition or a symptom associated with a deficiency or absence of endogenous testosterone, with a meal, a pharmaceutical composition comprising from about 50 mg to about 300 mg of testosterone undecanoate and a pharmaceutically acceptable carrier said method providing an increase in serum free testosterone levels. For example, the method can increase serum free testosterone levels by more than 10%, 20%, 30%, 40%, 50%, 70%, 100%, 120%, 150% or 200% over baseline.

In one embodiment, a method for replacement therapy in a male for having a condition or a symptom associated with a deficiency or absence of endogenous testosterone is provided, said method comprising: orally administering to a male having a condition or a symptom associated with a deficiency or absence of endogenous testosterone, with a meal, a pharmaceutical composition comprising from about 50 mg to about 300 mg of testosterone undecanoate and a pharmaceutically acceptable carrier said method providing a decrease in SHBG levels. For example, the method can decrease SHBG levels by more than 1%, 2%, 3%, 4%, 5%, 7%, 10%, 12%, 15%, 20%, 25% or 30% compared to baseline.

In one embodiment, a method for replacement therapy in a male for having a condition or a symptom associated with a deficiency or absence of endogenous testosterone is provided, said method comprising (1) identifying a male having (a) a deficiency or absence of endogenous testosterone and (b) one or more psychiatric disorders; (2) orally administering to a male having a condition or a symptom associated with a deficiency or absence of endogenous testosterone, with a meal, a pharmaceutical composition comprising from about 50 mg to about 300 mg of testosterone undecanoate and a pharmaceutically acceptable carrier, and (3) said method providing an improvement in one or more measures of quality of life Alternatively, the method involves treating a male having one or more psychiatric disorders by orally administering, with a meal, a pharmaceutical composition comprising from about 50 mg to about 300 mg of testosterone undecanoate and a pharmaceutically acceptable carrier. The method described in this embodiment, in some specific aspects, can improve a quality of life measure such as vitality, mental health, weekly negative mood, mental component summary or a combination thereof as measured by the SF-36, or an analogous measure. In another aspect, the method can treat one or more psychiatric disorders chosen from anxiety, stress, OCD, PTSD, ADD, ADHD, executive dysfunction, depression, depressive disorder, dysthymic disorder, major depressive disorder, bipolar disorder, bipolar II disorder, paranoia, mental disorder, alcohol abuse, alcoholism, nicotine dependence and tobacco use disorder.

Typically, the unit dosage forms contain an amount of TU as described herein and one or more pharmaceutically acceptable carriers e.g., additives. In one aspect, the unit dosage form is a tablet or capsule. In one aspect, the unit dosage form has a pharmaceutically acceptable carrier lipophilic additive, a hydrophilic additive, a solidifying agent or a combination thereof. In one aspect, the unit dosage form when tested using a USP type 2 apparatus in about 1000 mL 8% Triton X100 solution in water at 37.0±0.5 at 100 rpm releases at least 60% at 15 minutes and less than 100% at 15 minutes. In one aspect, the unit dosage form has a pharmaceutically acceptable carrier lipophilic additive, a hydrophilic additive, a solidifying agent or a combination thereof. In one aspect, the unit dosage form when tested using a USP type 2 apparatus in about 1000 mL 8% Triton X100 solution in water at 37.0±0.5 at 100 rpm releases less than 90% at 30 minutes and greater than 90% at 120 minutes. In one aspect, the unit dosage form when tested using a USP type 2 apparatus in about 1000 mL 8% Triton X100 solution in water at 37.0±0.5 at 100 rpm releases greater than 90% at 30 minutes. The unit dosage forms usually contain a lipophilic additive although this is not absolutely required. The dosage forms can also contain a hydrophilic additive, a solidifying agent, or one or more other additives. The pharmaceutically acceptable carriers a selected such that the oral dosage form is pharmaceutically equivalent, bioequivalent or both, in respect to serum testosterone levels to a capsule with the fill containing from 50 mg to 350 mg TU (e.g., 75 or 112.5 mg TU) at about 15% loading, 63% Maisine 35-1, 16% Cremophor RH 40 and 6% PEG 8000). According to some aspects, the formulation according to this embodiment, when administered twice a day, with food, to a male in need of testosterone replacement (e.g., having testosterone deficiency or a symptom thereof) will have an improvement in a measure of QOL, sexual function or a combination thereof. In respect to bioequivalent formulations, in one aspect, bioequivalent refers to steady state. In another aspect, bioequivalent refers to single dose. In yet another aspect, bioequivalent refers to food effect bioequivalent. Food effect bioequivalent refers to bioequivalence of the formulation under low fat versus standard fat meals, standard fat versus high fat meals, low fat versus high fat meals or a combination thereof. According to this embodiment, the method and composition is capable of providing an improvement in one or more quality of life measures or sexual domain function. In one aspect, the improvement in quality of life or sexual domain function is measurable by a questionnaire. In a specific aspect, the quality of life measurement is measurable by the SF-36 questionnaire. In another specific aspect, the quality of life measurement is measurable by the SF-36 questionnaire and is physical functioning, role physical, vitality, social functioning, mental health, physical component, mental component or combination thereof. In again another aspect, the improvement is sexual domain function is libido, erection, ejaculation, orgasm, satisfaction or a combination thereof. In still another aspect, the improvement in sexual domain function is overall level of sexual desire, pleasure with partner, pleasure without partner, positive mood, less negative mood, sexual activity, full erections, maintained erections or a combination thereof. The improvements referred to in this paragraph can be in reference to baseline.

In one embodiment, specific examples of oral dosage forms that are bioequivalent, pharmaceutically equivalent or both, to a capsule with the fill containing from 50 mg to 350 mg TU (e.g., 75 or 112.5 mg TU) at about 15% loading, 63% Maisine 35-1, 16% Cremophor RH 40 and 6% PEG 8000) include tablets, capsules, sachets, lozenges, granules, powders, sprinkle, suspension, liquids or combinations thereof. In another embodiment, the dosage form is coated. In one embodiment, the solid composition can be a matrix. In one embodiment, the solid oral dosage form is a tablet or a capsule. In another embodiment oral dosage form is a multiparticulate oral dosage form. In another embodiment, the composition can be multiparticulate. Regardless of the type, the oral dosage forms or compositions can be formulated to provide immediate, modified, delayed, sustained, extended, and/or controlled release of the testosterone undecanoate. The immediate, modified, delayed, extended, pulsatile, and/or controlled release can be achieved by any method known in the art so long as it does not interfere with the function of the solid oral dosage forms. Non-limiting examples of such methods includes coatings, polymers, and the like. In one embodiment, the oral dosage form can be uncoated. In one embodiment the solid composition of the invention is a solid dispersion, solid solution, molecular dispersion, co-precipitate, amorphate, solidified suspension, admixture, eutectic mixture, melt extrude, drug-carrier complex, thermosetting system, or combinations thereof.

In some embodiments, the oral dosage forms the present invention can be manufactured as tablet or capsule dosage forms either by dry granulation methods, or by wet granulation methods. For example, testosterone undecanoate can be combined with one or more pharmaceutically acceptable carrier and blended to get a homogenous mixture which can be compressed into a tablet or disposed into a capsule. In another embodiment, the homogenous mixture can be kneaded with a binder solution to get a wet granulate mass which can be dried and sized, for example by passing through ASTM mesh #30. The resulting granules can be optionally blended with pharmaceutical aids such as diluents, lubricants, disintegrants etc., and disposed into capsules or compressed into tablets. In another particular case, the tablets can be coated. In one embodiment the tablet is a matrix tablet. In another embodiment, the tablet can be multi-layered tablet dosage form which can achieve release characteristics that can accommodate dose splitting.

The oral dosage forms can also be formulated using melt-extrusion processes alone or in combination with other known processes. For example, in one embodiment, an amount of testosterone undecanoate can be homogeneously combined with a sufficient amount of one or more carrier substances prior to undergoing extrusion. The carrier suitable for the compositions of this invention, specifically melt extrusion process, can be lipophilic or hydrophilic carrier. Combinations of lipophilic and hydrophilic carriers may also be used.

The terms “melt” and “melting” should be interpreted broadly, and include not only the alteration from a solid state to a liquid state, but can also refer to a transition to a glassy state or a rubbery state in which it is possible for one component of the mixture to get embedded more or less homogeneously into the other. In particular cases, one component can melt and the other component(s) can dissolve in the melt, thus forming a solution which, upon cooling, may form a solid composition having advantageous properties. In another particular case, one component can melt and the other component(s) can suspend thus forming a suspension which upon cooling may form a solid suspension having advantageous properties.

The melt-extruded solid compositions used to make the oral dosage forms of the present disclosure can be granular, multiparticulates, pellets, beads, mini-tablets or tablets. The melt-extruded solids can be used alone as the solid oral dosage form or can be disposed into capsules or formed into tablets.

The carrier for a melt-extruded composition and/or dosage form can include, but is not limited to, carriers such as ethyl cellulose, cellulose acetate phthalates, glyceryl distearate, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, stearic acid, poly(methyl methacrylate) copolymer, polyacrylamide, aminoalkyl methacrylate copolymer, poly(methacrylic acid anhydride), and glycidyl methacrylate copolymers.

In one embodiment, the carrier for a melt-extruded oral dosage form can be one or more pharmaceutically acceptable polymers including, but not limited to, polyvinyl alcohol, polyvinyl pyrrolidone, polyethylene glycols having molecular weight of about 1000 to about 20,000, gelatin, carbomer, poloxamer, hydroxypropyl methyl cellulose; hydroxypropyl ethyl cellulose hydroxypropyl cellulose and carboxymethyl cellulose. It is noteworthy that some pharmaceutical carriers can be used in more than one manufacturing process, such as a wet milling or dry milling process as well as a melt extrusion process.

In certain embodiments, the at least one pharmaceutically acceptable carrier of any pharmaceutical composition provided herein comprises at least one hydrophilic carrier. In specific embodiments, the hydrophilic carrier is a hydrophilic triglyceride. In more specific embodiments, the hydrophilic triglyceride is a polyoxylated castor oil, or a polyoxylated hydrogenated castor oil. In some embodiments, any pharmaceutical composition provided herein has a lipophilic carrier (e.g., additive) or combination of lipophilic carriers. In certain embodiments, any pharmaceutical composition provided herein comprises a lipophilic carrier and less than 25%, 20%, 18%, 15% or 10% w/w or less than 5% w/w of a hydrophilic carrier (or hydrophilic surfactant).

In one embodiment, the lipophilic additive can include a lipophilic surfactant.

As used herein a surfactant is considered to be a lipophilic surfactant when it has an HLB value of 10 or less. Various lipophilic surfactants can be used including, but not limited to mono-, di-glycerides of fatty acids like glyceryl monolinoleate (e.g. Maisine® 35-1), mono- and di glycerides of caprylic, capric acid (e.g. Capmul® MCM), glyceryl monooleate, reaction mixtures of alcohols or polyalcohols with a variety of natural and/or hydrogenated oils such as PEG-5 hydrogenated castor oil. PEG-7 hydrogenated castor oil, PEG-9 hydrogenated castor oil, PEG-6 corn oil (e.g. Labrafil® M 2125 CS). PEG-6 almond oil (e.g. Labrafil® M 1966 CS), PEG-6 apricot kernel oil (e.g. Labrafil® M 1944 CS), PEG-6 olive oil (e.g. Labrafil® M 1980 CS). PEG-6 peanut oil (e.g. Labrafil® M 1969 CS), PEG-6 hydrogenated palm kernel oil (e.g. Labrafil® M 2130 BS), PEG-6 palm kernel oil (e.g. Labrafil® M 2130 CS), PEG-6 triolein (e.g. Labrafil® M 2735 CS), PEG-8 corn oil (e.g. Labrafil® WL 2609 BS). PEG-20 corn glycerides (e.g. Crovol® M40), PEG-20 almond glycerides (e.g. Crovol® A40), lipophilic polyoxyethylene-polyoxypropylene block co-polymers (e.g. Pluronic® L92, L101. L121 etc.); propylene glycol fatty acid esters, such as propylene glycol monolaurate (e.g. Lauroglycol FCC), propylene glycol ricinoleate (e.g. Propymuls), propylene glycol monooleate (e.g. Myverol P-O6), propylene glycol dicaprylate/dicaprate (e.g. Captex® 200), and propylene glycol dioctanoate (e.g. Captex® 800), propylene glycol mono-caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate: propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters. PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, and myristic acid; menthol, menthol derivatives, lecithin, phosphatidyl choline, bile salts, and the like, and mixtures thereof. It is important to note that some lipophilic surfactants may also function as the solubilizer component of the compositions and oral dosage forms.

In one embodiment, the lipophilic surfactant can be selected from the group consisting of glyceryl monolinoleate (e.g. Maisine® 35-1), mono- and di glycerides of caprylic, capric acid (e.g. Capmul® MCM), glyceryl monooleate, propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil. PEG-7 hydrogenated castor oil, PEG-9 hydrogenated castor oil, PEG-6 corn oil, PEG-6 almond oil, PEG-6 apricot kernel oil, PEG-6 olive oil. PEG-6 peanut oil, PEG-6 hydrogenated palm kernel oil, sorbitan monolaurate (e.g. Arlacel 20), sorbitan monopalmitate, sorbitan monooleate, sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof. In some embodiments, the lipophilic surfactants can comprise at least about 10, 20, 30, 40, 50, 60, 70, 80, or 90 wt % of the total pharmaceutically acceptable carrier. It should be noted that the combinations of two or more lipophilic surfactants from the same or different classes therein are also within the scope of this invention and are together can be referred to as the lipophilic surfactant, unless otherwise stated.

In one embodiment, the composition/dosage form has a hydrophilic additive or a hydrophilic additive which can be a hydrophilic surfactant. A surfactant is considered to be a hydrophilic surfactant when it has an HLB value of greater than 10. Non-limiting examples of hydrophilic surfactants include non-ionic surfactants, ionic surfactants and zwitterionic surfactants. Specifically the hydrophilic surfactants suitable for the current invention include, but not limited to alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di-ester mixtures; polysorbates, polyethylene glycol derivatives of tocopherol and the like It should be noted that the combinations of two or more hydrophilic surfactants from the same or different classes are within the scope of this invention and are together can be referred to as the hydrophilic surfactant unless explicitly specified. In one embodiment, the hydrophilic additive can be a hydrophilic surfactant. Non-limiting examples of hydrophilic surfactants can include PEG-8 caprylic/capric glycerides, lauroyl macrogol-32 glyceride, stearoyl macrogol glyceride. PEG-40 hydrogenated castor oil, PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, and mixtures thereof.

Suitable additives utilized in various embodiments described herein include, by way of non-limiting example, adsorbing agents, anti-adherents, anticoagulants, antifoaming agents, antioxidants, anti-caking agents, anti-static agents, binders, bile acids, bufferants, bulking agents, chelating agents, coagulants, colorants, co-solvent, opaquants, congealing agents, coolants, cryoprotectants, diluents, dehumidifying agents, desiccants, desensitizers, disintegrants, dispersing agents, enzyme inhibitors, glidants, fillers, hydrating agent, super disintegrants, gums, mucilages, hydrogen bonding agents, enzymes, flavorants, humectants, humidifying agents, lubricant oils, ion-exchange resins, lubricants, plasticizers, pH modifying agents, preservatives, solidifying agent, solvents, solubilizers, spreading agent sweeteners, stabilizers, surface area enhancing agents, suspending agent, thickeners, viscosity increasing agents, waxes and mixtures thereof.

Some non-limiting examples of the additives suitable for the present disclosure may be: alcohols and/or polyols (e.g., ethanol, isopropanol, butanol, benzyl alcohol, ethylene glycol, propylene glycol, glycerol, sorbitol, mannitol, dimethyl isosorbide, polyethylene glycol, fatty acid alcohol, vinyl alcohol polypropylene glycol, polyvinylalcohol, tocopherols, cellulose cyclodextrins, other derivatives, forms, mixtures thereof, or the like); ethers of polyethylene glycols having an average molecular weight of about 200 to about 20,000 (e.g. tetrahydrofurfuryl alcohol PEG ether, methoxy PEG, or the like); amides (e.g. 2-pyrrolidone, 2-piperidone, 8-caprolactam. N-alkylpyrrolidone, N-hydroxyalkylpyrrolidone, N-alkylpiperidone. N-alkylcaprolactam, dimethylacetamide, polyvinylpyrrolidone and the like.); esters (e.g. ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, 8-caprolactone and isomers thereof, 6-valerolactone and isomers thereof, gamma-butyrolactone and isomers thereof; and other additives known in the art, such as dimethyl acetamide, dimethyl isosorbide. N-methylpyrrolidones, monooctanoin, diethylene glycol monoethyl ether, or the like): amino acids (e.g. p-aminobenzamidine, sodium glycocholate) mesylate: amino acids and modified amino acids (e.g. aminoboronic acid derivatives and n-acetylcysteine; peptides and modified peptides (e.g. bacitracin, phosphinic acid dipeptide derivatives, pepstatin, antipain, leupeptin, chymostatin, elastin, bestatin, phoshporamindon, puromycin, cytochalasin potatocarboxy peptidase inhibitor, amastatin, or the like); polypeptide protease inhibitors: mucoadhesive polymers (e.g. polyacrylate derivatives, chitosan, cellulosics, chitosan-EDTA, chitosan-EDTA-antipain, polyacrylic acid, carboxymethyl cellulose etc.) or the like; or combinations thereof.

Some more examples of suitable additives for compositions and/or dosage forms described herein include, by way of non-limiting example, talc, magnesium stearate, silica (e.g. fumed silica, micronized silica, magnesium aluminum silicate etc.) and/or derivatives, polyethylene glycols, surfactants, waxes, oils, cetyl alcohol, polyvinyl alcohol, stearic acid, stearic acid salts, stearic acid derivatives, starch, hydrogenated vegetable oils, hydrogenated castor oils, sodium benzoate, sodium acetate, leucine. PEG, alkyl sulfate salts; acetylated monoglycerides: long-chain alcohols; silicone derivatives; butylated hydroxy toluene (BHT), butylated hydroxyl anisole (BHA), gallic acid, propyl gallate, ascorbic acid, ascorbyl palmitate, 4-hydroxymethyl-2,6-di-tert-butyl phenol, dry starch, dry sugars, polyvinyl pyrrolidones, starch paste, methacrylic copolymers, bentonite, sucrose, polymeric cellulose derivatives, shellac, sugar syrup; corn syrup; polysaccharides, acacia, tragacanth, guar gum, xanthan gums: alginates; gelatin; gelatin hydrolysate; agar, sucrose; dextrose; PEG, vinyl pyrrolidone copolymers, poloxamers; pregelatinized starch, sorbitol, glucose); acetic acid, hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, vinegar, pharmaceutically acceptable bases, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamin; salt of a pharmaceutically acceptable cation and an anion; EDTA and EDTA salts; titanium dioxide, food dyes, lakes, natural vegetable colorants, iron oxides, silicates, sulfates, magnesium hydroxide and aluminum hydroxide; halogenated hydrocarbons, trichloroethane, trichloroethylene, dichloromethane, fluorotrichloromethane, diethylether, trehalose, phosphates, citric acid, tartaric acid, gelatin, dextran and mannitol, lactose, mannitol, sodium chloride, potassium chloride, spray-dried lactose, hydrolyzed starches, directly compressible starch, microcrystalline cellulose, cellulosic derivatives, sorbitol, sucrose, sucrose-based materials, calcium sulfate, dibasic calcium phosphate, dextrose, croscarmellose sodium, starch, starch derivatives, clays, gums, cellulose, cellulose derivatives, alginates, crosslinked polyvinylpyrrolidone, sodium starch glycolate and microcrystalline cellulose, magnesium oxide, magnesium carbonates; desensitizers, spray-dried flavors, essential oils, ethyl vanillin, styrene/divinyl benzene copolymers, quaternary ammonium compounds, polyethylene glycol, citrate esters (such as triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl sebacate, ascorbic acid, boric acid, sorbic acid, benzoic acid, and salts thereof, parabens, phenols, benzyl alcohol, and quaternary ammonium compounds; alcohols, ketones, esters, chlorinated hydrocarbons water, sweeteners (e.g. maltose, sucrose, glucose, sorbitol, glycerin and dextrins, aspartame, saccharine, saccharine salts, glycyrrhizin), viscosity modifiers, sugars, polyvinylpyrrolidone, cellulosics, polymers, gums and/or alginates.

In one embodiment, additives may also be materials such as proteins (e.g., collagen, gelatin. Zein, gluten, mussel protein, lipoprotein); carbohydrates (e.g., alginates, carrageenan, cellulose derivatives, pectin, starch, chitosan); gums (e.g., xanthan gum, gum Arabic); spermaceti: natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based polymers (e.g., ethyl cellulose, methyl cellulose, microcrystalline cellulose, sodium carboxymethyl cellulose, hydroxypropylmethyl cellulose, ethyl cellulose, hydroxypropyl cellulose, HPMC acid succinates, cellulose acetate, cellulose nitrate, cellulose acetate butyrate, cellulose acetate trimellitate, carboxymethylethyl cellulose, hydroxypropylmethyl cellulose phthalate), shellacs; inorganics, such as dicalcium phosphate, hydroxyapatite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar. Non-limiting examples of compounds (e.g., additives) that can be used as at least a part of the pharmaceutically acceptable carrier include without limitation celluloses; dextrins, gums, carbomers, methacrylates, sugars, lactoses, inorganic carbonates, oxides, chlorides, sulphates and the like; salts of calcium; salts of magnesium; salts of fatty acids: inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

In one embodiment, the additive may include at least one component selected from celluloses, dextrins, gums, carbomers, methacrylates, inorganic carbonates, salts of calcium, salts of magnesium, fatty acids, fatty acid esters, gelatin, lactoses, polyethylene glycol, polyethylene oxide co-polymers, silicates, partially hydrogenated vegetable oils, fully hydrogenated vegetable oils, waxes, antioxidants, tocopherol, sugar stearates, starches, shellac, resins, proteins, and combinations thereof.

In another embodiment, the additive may include at least one component selected from celluloses, dextrins, gums, carbomers, methacrylates, sugars, lactoses, inorganic carbonates, salts of calcium, salts of magnesium, salts of fatty acids, inorganic and organic acids, bases and salts, propylene glycol, glycerols, fatty acids, fatty alcohols, fatty acid esters, glycerol esters, mono-glycerol esters of fatty acids, di-glycerol esters of fatty acids, mixtures of mono-glycerol and di-gylcerol esters of fatty acids, omega oils, waxes, alcohols, gelatin, polyethylene glycol, polyethylene oxide co-polymers, silicates, antioxidants, tocopherol, sugar stearates, starches, shellac, resins, proteins, acrylates, methyl copolymers, polyvinyl alcohol, starch, phthalates, and combinations thereof.

Non-limiting examples of additives as release modulators that may be used include lipophilic resins; ethyl cellulose (EC), methylethyl cellulose (MEC), carboxymethyl ethylcellulose (CMEC), hydroxyethyl cellulose (HEC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose phthalate (HPMCP), hydroxypropyl methyl cellulose acetate succinate (HPMCAS), hydroxypropyl methyl cellulose acetate trimellitate (HPMCAT), ion-exchange resin; poloxamers; and ethylhydroxy ethylcellulose (EHEC) tocopherol; shellac; and combinations thereof. Non-limiting examples of lipidic lipophilic release modulators include fatty acids; mono-, di-, tri-esters of fatty acids with glycerol; sucrose esters with fatty acids; cetyl alcohol; stearic acid; glyceryl monostearate; glyceryl distearate; glyceryl tristearate; glyceryl palmitostearate; hydrogenated castor oil; butyl and glycol esters of fatty acids; oleic acid; cetyl alcohol; stearyl alcohol; cetostearyl alcohol; hydrogenated vegetable oil; waxes; bees wax: lard: omega fatty acid esters; hydrogenated soybean oil; hydrogenated vegetable oil; hydrogenated cottonseed and castor oil; partially hydrogenated soybean oil; partially hydrogenated castor oil; partially soy and cottonseed oil; phospholipids; hydrogenated oils, and their derivatives and combinations thereof.

In some embodiments, the pharmaceutical composition (e.g., oral dosage form) provided herein is formulated, e.g., with a viscosity enhancing agent or solidifying agent, to provide a solid, a semi-solid, a gel, a jelly, a paste, or the like. In some embodiments, the oral dosage form is a liquid. Non-limiting examples of formulations (and for use in the methods described herein) are given in the tables below.

TABLE A

| | Capsule A1 | Capsule A2 |
|---|---|---|
| Component | % w/w | % w/w |
| Testosterone Undecanoate (50-350 mg) | 1-50 | 10-30 |
| Hydrophilic Carrier | 0-90 | 0-30 |
| Lipophilic Carrier | 1-90 | 40-70 |
| Other Additive(s) | 0-20 | 0-10 |

TABLE B

| | Capsule B1 | Capsule B2 |
|---|---|---|
| Component | % w/w | % w/w |
| Testosterone Undecanoate | 1-50 | 10-30 |
| (75, 112.5, 150, 225, or 300 mg) | | |
| Hydrophilic Carrier | 0-90 | 0-30 |
| Lipophilic Carrier | 1-90 | 40-70 |
| Other Additive(s) | 0-20 | 0-10 |

TABLE C

| | Capsule C1 | Capsule C2 |
|---|---|---|
| Component | % w/w | % w/w |
| Testosterone Undecanoate | 5-40 | 10-30 |
| (75, 112.5, 150, 225, or 300 mg) | | |
| Hydrophilic Carrier | 0-90 | 0-30 |
| Lipophilic Carrier | 1-90 | 40-70 |
| Solidifying Agent | 1-20 | 3-10 |

TABLE D

| | Capsule D1 | Capsule D2 |
|---|---|---|
| Component | % w/w | % w/w |
| Testosterone Undecanoate | 5-40 | 10-30 |
| (75, 112.5, 150, 225, or 300 mg) | | |
| Cremophor RH 40 | 0-90 | 0-30 |
| Glyceryl Monolinoleate | 1-90 | 40-70 |
| PEG 8000 | 1-20 | 3-10 |

TABLE E

| | Capsule E1 | Capsule E2 |
|---|---|---|
| Component | % w/w | % w/w |
| Testosterone Undecanoate | 10-25 | 13-23 |
| (75, 112.5, 150, 225, or 300 mg) | | |
| Cremophor RH 40 | 0-25 | 10-20 |
| Glyceryl Monolinoleate | 30-90 | 40-70 |
| Solidifying agent | 0-20 | 3-10 |

Exemplary bioequivalent and/or pharmaceutically equivalent formulations having the drug label as described herein or for use in the method included herein can include those described about in Tables A-E or e.g., in reference to a formulation having 50 mg to 350 mg testosterone undecanoate (e.g., in one aspect either 75 mg or 112.5 mg TU) at about 15% loading, about 63% Maisine 35-1, about 16% Cremophor RH 40 and about 6% PEG 8000 as follows.
(1) Varying the loading of testosterone undecanoate.
(2) Varying the amount of Maisine 35-1, exchanging Maisine 35-1 for another lipophilic additive (in varying amounts) or including one or more additional lipophilic additives in addition to Maisine 35-1 (in varying amounts). In one aspect, the other lipophilic additive is a fatty acid or ester thereof such as a fatty acid glyceride (e.g., mono-, di- or tri-glyceride) or a reaction mixture of a fatty acid glyceride with an alcohol e.g., polyethylene glycol, or a combination thereof.
(3) Varying the amount of Cremophor RH 40, exchanging Cremophor RH 40 for another hydrophilic additive (in varying amounts) or including one or more additional hydrophilic additives in addition to Cremophor RH 40 (in varying amounts). In one aspect, the other hydrophilic additive is a polyoxylated hydrogenated vegetable oil. In another aspect, the other hydrophilic additive is an ionic or non-ionic surfactant.
(4) Varying the amount of PEG 8000 (or not include PEG 8000), exchanging PEG 8000 for another solidifying agent (in varying amounts) or including one or more additional solidifying agents in addition to PEG 8000 (in varying amounts). In one aspect, the other solidifying agent is a high molecular weight PEG (2000 mw or more), a phytosterol or ester thereof, solid (at room temperature) fatty acids or mono- or di-glycerides and the such.
(5) Including one or more additional pharmaceutically acceptable carriers (in varying amounts); or
(6) A combination of one or more of (1)-(5).

In one embodiment, a method is provided said method comprising administering as a replacement therapy in a male having testosterone deficiency a pharmaceutical composition having about 75 mg, 112.5 mg, 150 mg, 225 mg or 300 mg TU at about 15% loading, about 63% Maisine 35-1, about 16% Cremophor RH 40 and about 6% PEG 8000 twice daily, with a meal, at a total daily dose of about 300 mg, 450 mg or 600 mg TU for at least 1, 2, 3, or 4 weeks to provide serum testosterone levels in the range of about 300 ng/dL to about 1140 ng/dL and an improvement in one or more measures of quality of life or sexual function (e.g., sexual domain function) over baseline. According to one aspect of this embodiment, the male commences treatment with a starting daily dose of about 450 mg of TU (e.g., 225 mg in the morning and 225 mg in the evening). After at least 1 week on this dosing regimen a serum testosterone level measurement is made after single dose administration (e.g., morning dose). If the measured serum testosterone level is too high, then the subject is switched or titrated to the 300 mg per day dosing regimen or if it is too low then the subject is switched to the 600 mg per day dosing regimen. Preferably the subjects serum testosterone level, as determined by C_{avg,24h} is maintained in the range of about 300 ng/dL to 1140 ng/dL, and more preferably about 300 ng/dL to about 800 ng/dL with minimal to no Cₘₐₓ excursion above 2500 ng/dL or 3000 ng/dL. It is noted that the drug label can indicate (or method involve) that the oral testosterone replacement therapy (testosterone undecanoate containing oral dosage form) is taken (1) “WITH A MEAL” or (2) “WITH MEAL. BUT NOT ON EMPTY STOMACH” or (3) “WITH FAT CONTAINING FOOD” not specifying fat content. In an alternative, the drug label may indicate (or method may involve) the oral testosterone replacement therapy (testosterone undecanoate containing oral dosage form) is taken “WITH MEAL, BUT NOT LOW FAT”. In an alternative, the drug label may indicate (or method may involve) the oral testosterone replacement therapy (testosterone undecanoate containing oral dosage form) is taken “WITH MEAL, BUT NOT HIGH FAT”. In an alternative, the drug label may indicate (or method may involve) the oral testosterone replacement therapy (testosterone undecanoate containing oral dosage form) is taken “WITH STANDARD OR NORMAL MEAL”. According to this embodiment, the improvement in QOL, in one aspect, is an improvement in role physical, vitality, social functioning, mental health, mental component summary or a combination thereof as assessed using the SF-36 questionnaire or any other appropriate corollary. In a more specific aspect, the improvement in QOL is in 2, 3, 4, or 5 measures indicated in the previous sentence. According to this embodiment, the improvement in sexual function, in one aspect, is an improvement in overall level of sexual desire, pleasure with partner, pleasure without partner, positive mood, less negative mood, sexual activity, full erections, maintained erections or a combination thereof. In a more specific aspect, the improvement in sexual function is in 2, 3, 4, or 5 measures indicated in the previous sentence. In one aspect, the formulation is as in the previous paragraph and bioequivalent, therapeutically equivalent or a combination thereof to that described in this paragraph.

The ordinary skilled artisan, in view of the instant disclosure recognizes that other formulations can be designed that are bioequivalent to those described herein or that can be designed to achieve the same outcomes or better in respect to the improvements in quality of life and sexual domain function. As such, the invention should not be considered to be limited to any specific embodiments, examples, or disclosure described herein.

### EXAMPLES

The following examples are provided to promote a more clear understanding of certain embodiments of the present invention, and are in no way meant as a limitation thereon.

### Example 1: Clinical Study

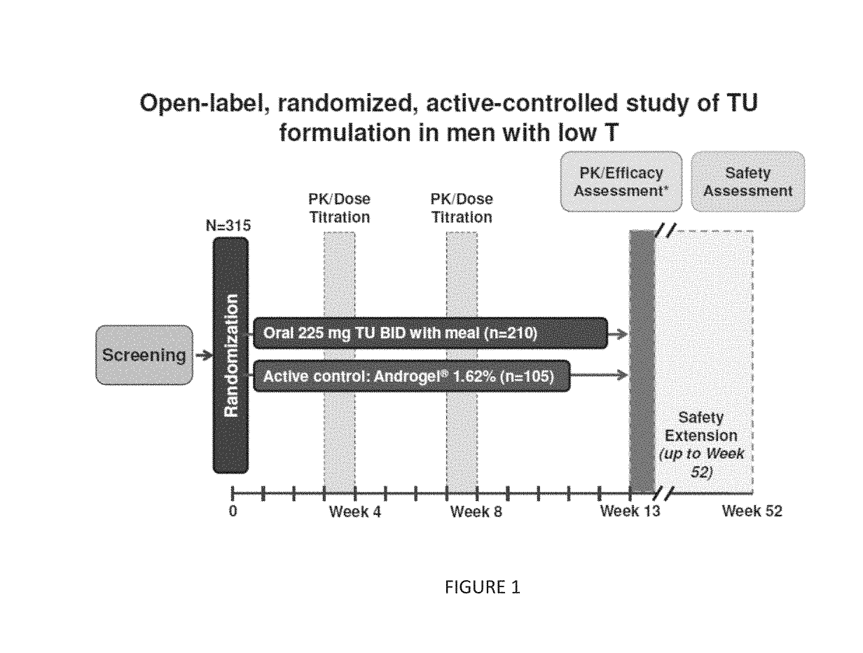

A clinical trial was designed and implemented as diagrammed in FIG. 1 using a formulation having 50 mg to 350 mg testosterone undecanoate (e.g., 75 mg or 112.5 mg TU) at about 15% loading, about 63% Maisine 35-1, about 16% Cremophor RH 40 and about 6% PEG 8000 with an initial daily dose of 450 mg per day (e.g., 225 mg TU twice a day with a meal). Subjects were up titrated to 600 mg per day, down titrated to 300 mg per day or maintained at 450 mg per day depending on the serum testosterone pharmacokinetic results at weeks 4 and 8.

Pharmacokinetic/efficacy results for the oral TU arm are shown below.

| Measure | FDA Threshold | Frequency* |
|---|---|---|
| % of subjects with | ≥75% | 88.2 |
| C_{avg,24 h} within 300-1140 ng/dL | | |
| 95% CI lower bound0 (%) | ≥65% | 81.9 |

Serum testosterone levels were reliably restored and maintained in the eugonadal range (300-1140 ng/dL) for more than 85% of hypogonadal men with mean (CV) C_{avg,24h} of 447 (37%) ng/dL in the efficacy dataset (efficacy population (n=152)).

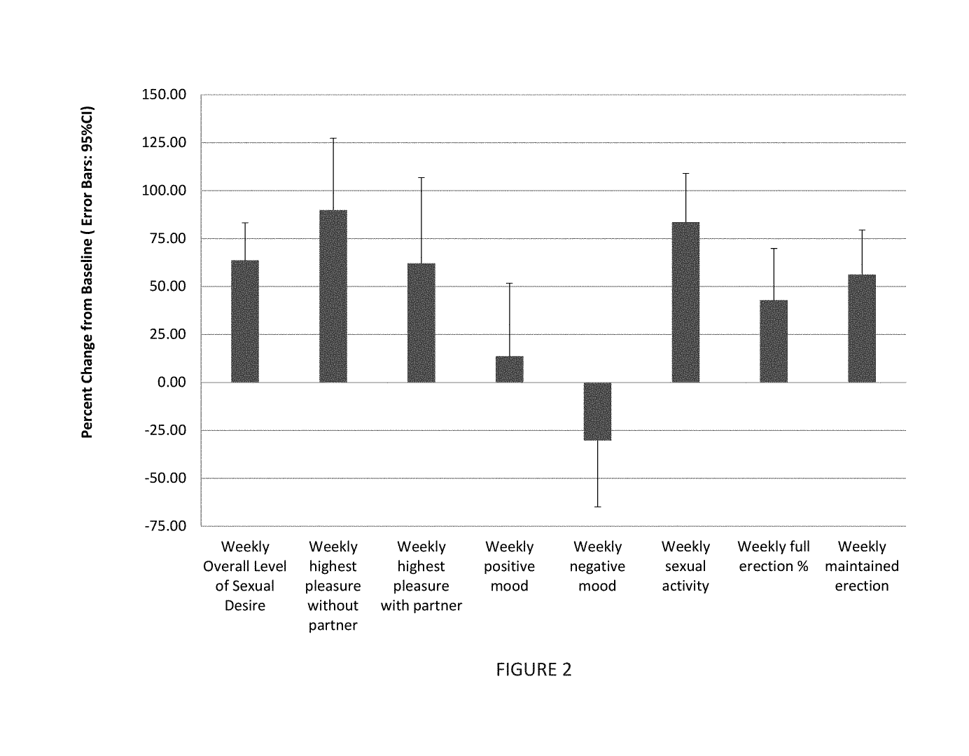

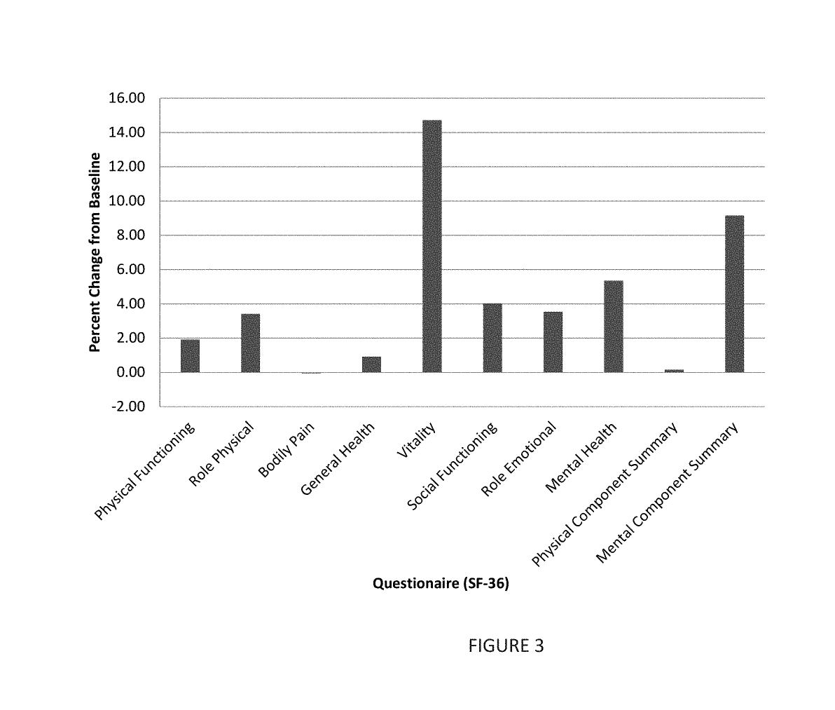

During this trial a quality of life questionnaire (see FIG. 3) and a sexual function questionnaire (see FIG. 2) was administered at the beginning and end of the clinical trial. Results are summarized below.
The results shown in FIG. 2 are statistically significant changes from baseline for each parameter with P values less than 0.001 for each parameter. Thus, the oral testosterone replacement therapy substantial and significantly improved sexual domain function compared to baseline reporting at the beginning of the trial. In addition, the oral composition and treatment as described herein showed statistically significant improvements over the Androgel arm for several measures including reducing negative mood and improving maintained erection.
The results shown in FIG. 3 are statistically significant changes from baseline for role physical (p<0.05), vitality (p<0.001), social functioning (p<0.05), mental health (p<0.05), and mental component summary (p<0.001) as assessed using the SF-36 questionnaire. For each of these items showing statistical significance, they were also unexpectedly better than the values for Androgel including statistically significant improvements on measures of mental health and mental component summary.

### Example 2: Bioequivalent Formulations and Oral Dosage Forms

The ordinary skilled artisan in view of these results can design and test formulations for bioequivalence in view of this disclosure.

Formulations for testing in the study as described in Example 1 (or another appropriate designed study like that required for filing an Abbreviated New Drug Application or ANDA) can be designed e.g., by:

(1) varying the loading of testosterone undecanoate;
(2) varying the amount of Maisine 35-1, exchanging Maisine 35-1 for another lipophilic additive (in varying amounts) or including one or more additional lipophilic additives in addition to Maisine 35-1 (in varying amounts);
(3) varying the amount of Cremophor RH 40, exchanging Cremophor RH 40 for another hydrophilic additive (in varying amounts) or including one or more additional hydrophilic additives in addition to Cremophor RH 40 (in varying amounts):
(4) varying the amount of PEG 8000 (or not include PEG 8000), exchanging PEG 8000 for another solidifying agent (in varying amounts) or including one or more additional solidifying agents in addition to PEG 8000 (in varying amounts);
(5) including one or more additional pharmaceutically acceptable carriers (in varying amounts); or
(6) a combination of one or more of (1)-(5).

Typically these formulations will be suited for soft gelatin or hard gelatin capsules.

Other dosage forms are also contemplated including tablets, sachets, lozenges, granules, powders, sprinkle, suspension, liquids or combinations thereof.

### Example 3: Clinical Trial to Show Bioequivalence

To determine if a Test formulation is bioequivalent to the formulation used in Example 1, a bioequivalence study is performed under the same or similar conditions. One of ordinary skill in the art can design and perform a bioequivalence study in view of the results presented in Example 1. Test formulations can be generated according to this disclosure or more particularly Example 2. The bioequivalence study is one that may support the filing of an Abbreviated New Drug Application at the US FDA or a similar application in jurisdictions outside of the United States with the appropriate regulatory agency.

### Example 4: Free Testosterone & SHBG Levels

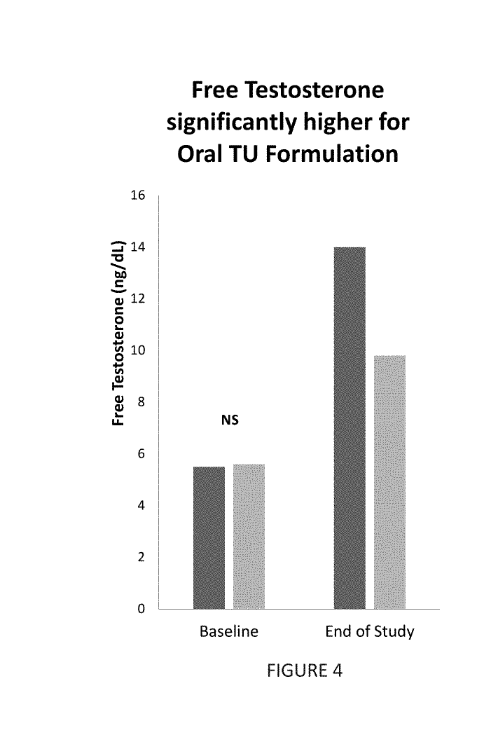

During the clinical trial as illustrated/described in Example 1, it was also found that free testosterone levels were significantly higher at the end of study as compared to baseline for the oral formulation described herein as well as compared to the Androgel arm (P=0.003) (see FIG. 4). In FIG. 4 the left bar in each pair is the oral treatment arm whereas the right bar is the Androgel treatment arm.

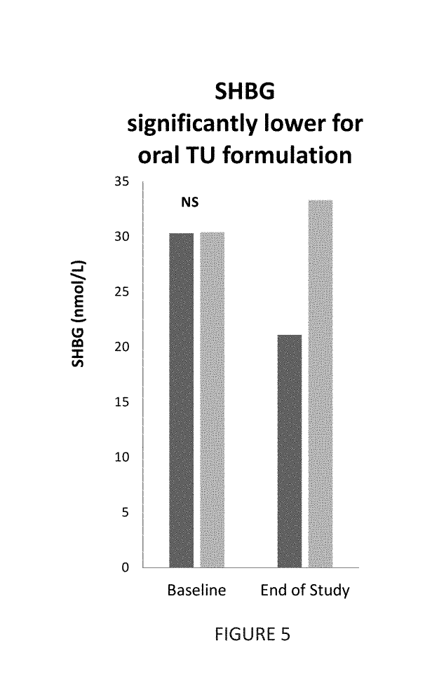

During the clinical trial as illustrated/described in Example 1, it was also found that SHBG were significantly lower at the end of study as compared to baseline for the oral formulation described herein as well as compared to the Androgel arm (P<0.0001) (see FIG. 5). In FIG. 5 the left bar in each pair is the oral treatment arm whereas the right bar is the Androgel treatment arm.

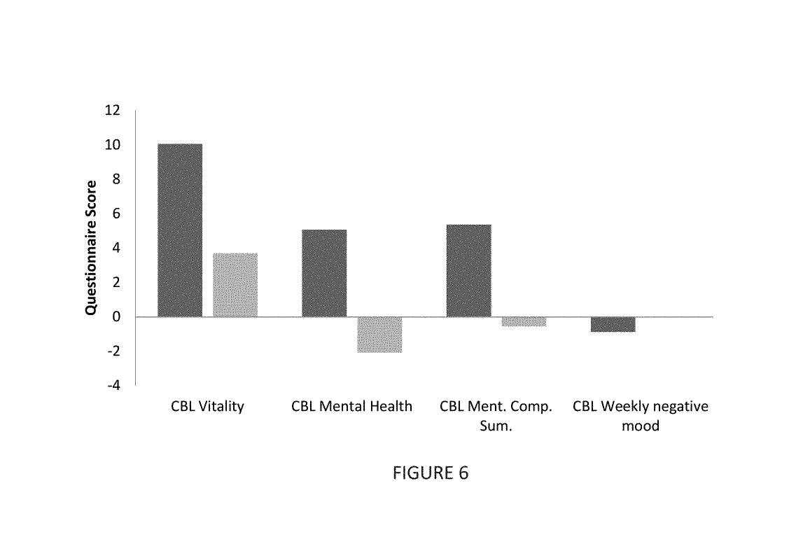

During the clinical trial as illustrated/described in Example 1, another interesting, unexpected finding was that patients having one or more psychiatric disorders at baseline had significant improvements in one or more quality of life measurements (as determined via the SF-36 questionnaire) after treatment with the oral formulation described herein as compared to treatment with Androgel as illustrated in FIG. 6. In FIG. 6, the darker colored bars (left) in each pair correspond to oral treatment whereas the light colored bars (right) correspond to Androgel treatment.

The p-values in the chart above are 0.028 for vitality, 0.009 for mental health, 0.003 for weekly negative mood and 0.020 for mental component summary.

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

1. A method for replacement therapy in a male for having a condition or a symptom associated with a deficiency or absence of endogenous testosterone, said method comprising: orally administering to a male having a condition or a symptom associated with a deficiency or absence of endogenous testosterone, with a meal, a pharmaceutical composition comprising from about 50 mg to about 300 mg of testosterone undecanoate and a pharmaceutically acceptable carrier said method providing an improvement in one or more quality of life measures or sexual domain function measures.

2. The method of claim 1 said improvement in quality of life or sexual domain function is measurable by a questionnaire.

3. The method of claim 1 said quality of life measurement is measurable by the SF-36 questionnaire.

4. The method of claim 1 said quality of life measurement is measurable by the SF-36 questionnaire and is physical functioning, role physical, vitality, social functioning, mental health, physical component, mental component or a combination thereof.

5. The method of claim 1 said sexual domain function is libido, erection, ejaculation, orgasm, satisfaction or a combination thereof.

6. The method of claim 1 said sexual domain function is overall level of sexual desire, pleasure with partner, pleasure without partner, positive mood, less negative mood, sexual activity, full erections, maintained erections or a combination thereof.

7. The method as in claim 1, said pharmaceutical composition is a unit dosage form having about 75 mg, about 112.5 mg, about 150 mg, about 225 mg, or about 300 mg of testosterone undecanoate.

8. The method as in claim 1 said pharmaceutical acceptable carrier selected to provide bioequivalent amounts of serum testosterone levels to said male for meals containing low, standard fat and high fat.

9. The method as in claim 1 said method providing a serum testosterone C_{avg,24h} in the range of 300 ng/dL to 1140 ng/dL.

10. The method as in claim 1, said administering is twice-a-day.

11. The method as in claim 1, said method comprising administering: (a) from 285 mg to about 625 mg of testosterone undecanoate per day; (b) about 300 mg per day; (c) about 450 mg per day; or (d) about 600 mg per day.

12. The method as in claim 1, said composition comprising a lipophilic additive.

13. The method as in claim 1, said composition comprising a hydrophilic additive.

14. The method as in claim 1, said pharmaceutical composition being pharmaceutically equivalent, bioequivalent or both to an oral pharmaceutical composition (1) having (a) about 75 mg or about 112.5 mg of testosterone undecanoate at about 15% loading and (b) about 63% Maisine 35-1, about 16% Cremophor RH 40 and about 6% PEG 8000 or (2) having (a) about 75 mg, about 112.5 mg, about 150 mg, about 225 mg or about 300 mg of testosterone undecanoate at about 15% loading and (b) about 63% Maisine 35-1, about 16% Cremophor RH 40 and about 6% PEG 8000.

15. The method as in claim 1, said method comprising administering the pharmaceutical composition as 2, 3, 4, 5, 6, 7, or 8 unit dosage forms per day.

16. A method for replacement therapy in a male for a condition or symptom associated with a deficiency or absence of endogenous testosterone, said method comprising: orally administering to a male having a condition or symptom associated with a deficiency or absence of endogenous testosterone, a pharmaceutical composition comprising from about 50 mg to about 300 mg of testosterone undecanoate and a pharmaceutically acceptable carrier said method providing an improvement in one or more quality of life measures.

17. The method of claim 16 said improvement in quality of life is measurable by a questionnaire.

18. The method of claim 16 said quality of life measurement is measurable by the SF-36 questionnaire.

19. The method of claim 16 said quality of life measurement is measurable by the SF-36 questionnaire and is physical functioning, role physical, vitality, social functioning, mental health, physical component, mental component or a combination thereof.

20. The method as in claim 16, said pharmaceutical composition being pharmaceutically equivalent, bioequivalent or both to an oral pharmaceutical composition (1) having (a) about 75 mg, about 112.5 mg, about 150 mg, about 225 mg or about 300 mg of testosterone undecanoate at about 15% loading and (b) about 63% Maisine 35-1, about 16% Cremophor RH 40 and about 6% PEG 8000 or (2) having (a) about 75 mg or about 112.5 mg of testosterone undecanoate at about 15% loading and (b) about 63% Maisine 35-1, about 16% Cremophor RH 40 and about 6% PEG 8000.

21-42. (canceled)
